SEQUENCE LISTING (6 sequences)

SEQ ID NO: 1 - protein, 153 aa, Artificial Sequence
  Description of Artificial Sequence Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Artificial Sequence [sequence omitted]

SEQ ID NO: 3 - protein, 132 aa, Artificial Sequence
  Description of Artificial Sequence Synthetic polypeptide
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 133 aa, Artificial Sequence
  Description of Artificial Sequence Synthetic polypeptide
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 5 - protein, 512 aa, Artificial Sequence
  Description of Artificial Sequence Synthetic polypeptide
MQTSPTGILPTTSNSISTSEMTWKSSFPEFARYTTPEDTTPEPGEDPRVTRHSGQNHLKEMAISVLEARACAAAGQTVSVVALHDDMENQPLIGIQSTAIPEVATRMQSFGMKIVGYDPIISPEVAIIQVSPKDIQLTIFPSKSVKEGDTVKASKKGMWSEGNSSHTIRDLKYTIETSIPSVSNALNWKEFSFIQSTLGYVLRTAAYVNAIEKIFKVYNEAGVTFTSWIHCWKYLSVQSQLFRGSSLLFRRSNFTVDCSKAGNDMLLVGVHGPRTPALGSLALMIWLMTTPHSHETEQKRLLPGFKGVKGHSGIDGLKGQPGAQGVAVQKLNLQNLVILQAPENLTLSNLSESDRNKESSDQTSVNMNGLENKISYLLPFYPPDEALEIGLELNSSALPPTILPQAPSGPSYATYLQPAQAQMLTPKPLRRNNSYTSYIMAICGMPLDSFRVIQTSKYYMRDVIAIESAWLLELAPHIHRAGGLFVADAIQVGFGRIGKHFGYPYDVPDYAS

SEQ ID NO: 6 - protein, 9 aa, Artificial Sequence
  Description of Artificial Sequence Synthetic peptide
SPSYVYHQF

(19) United States
(12) Patent Application Publication
(10) Pub. No.: US 2019/0275133 A1
(43) Pub. Date: Sep. 12, 2019

(54) IMMUNOTHERAPEUTIC TUMOR TREATMENT METHOD

(71) Applicant: Nektar Therapeutics, San Francisco, CA (US)

(72) Inventors: Deborah H. Charych, Albany, CA (US); Jonathan Zalevsky, Orinda, CA (US); Willem Overwijk, San Francisco, CA (US); Meenu Sharma, Houston, TX (US); Adi Diab, Houston, TX (US); Patrick Hwu, Houston, TX (US)

(21) Appl. No.: 16/349,227

(22) PCT Filed: Nov. 9, 2017
(86) PCT No.: PCT/US17/60911

§ 371 Date: May 10, 2019

Related U.S. Application Data
(60) Provisional application No. 62/420,442, filed on Nov. 10, 2016.
(60) Provisional application No. 62/582,852, filed on Nov. 7, 2017.

(51) Int. Cl. A61K 39/00; A61K 38/20; A61K 39/39; A61P 35/00

(52) U.S. Cl. CPC A61K 39/001192; A61K 38/2013; A61K 39/39; A61K 2039/51; A61K 2039/545; A61K 2039/55533; A61P 35/00

(57) ABSTRACT

Provided herein are methods and compositions for treating a subject having cancer by administering to the subject a cancer vaccine accompanied by administration of a long acting IL-2Rαβ-biased agonist.

### CROSS REFERENCE TO RELATED APPLICATIONS

This application claims the benefit of priority under 35 U.S.C. § 119(e) to U.S. Provisional Patent Application No. 62/420,442, filed on Nov. 10, 2016, and to U.S. Provisional Patent Application No. 62/582,852, filed on Nov. 7, 2017, the disclosures of which are incorporated herein by reference in their entireties.

### FIELD

The instant application relates to (among other things) the field of immunotherapy, and in a particular aspect, cancer immunotherapy, and involves the treatment of an individual having cancer by administering to the individual a cancer vaccine, accompanied by administration of a long acting IL-2Rαβ-biased agonist.

### BACKGROUND

Therapeutic cancer vaccines represent a class of substances that work by stimulating or restoring a subject's immune system's ability to fight infections and disease. Therapeutic vaccines, as opposed to preventative or prophylactic vaccines, are used to treat an existing cancer by boosting the body's natural immune response against the cancer and represent a type of immunotherapy. Cancer treatment vaccines are designed to activate cytotoxic T cells and direct them to recognize and act against specific types of cancer or to induce production of antibodies that bind to molecules on the surface of cancer cells. However, producing effective therapeutic vaccines has proven to be a challenging endeavor, because the vaccine intervention must combat the body's immune system that is restrained by mechanisms that work to sustain the cancer. To be effective, a therapeutic cancer vaccine must not only stimulate a specific immune response against the intended target, but must also be powerful enough to overcome the barriers that cancer cells utilize to protect themselves from attack by killer T cells. Over the last several years there have been substantial efforts in developing therapeutic vaccines encompassing various platforms, however, only one vaccine, Provenge® (sipuleucal-T, an autologous vaccine), has received FDA approval to date. Therapeutic vaccines have been evaluated, for example, in patients with breast cancer, lung cancer, melanoma, pancreatic cancer, colorectal cancer, and renal cancer (Melero, I., et al., Nat Rev Clin Oncol, 2014, 11 (9), 509-524).

To improve immunization anticancer strategies, substances such as adjuvants can be added to vaccines to boost their ability to induce potent anticancer immune responses, although improved responses can often be partial and/or transient. Adjuvants for cancer vaccines can come from a variety of sources, such as bacteria, substances produced by bacteria, proteins, and synthetic or natural cytokines. Various substances including cytokines have been investigated for enhancing vaccine-induced antitumor activity. While some cytokines appear to function as effective adjuvants, others have been found to be surprisingly ineffective in modulating vaccine effectiveness. Cytokines used in cancer treatment vaccines include, for example, IL-2, interferon-alpha, and granulocyte-macrophage colony stimulating factor (GM-CSF).

Although there have been substantial efforts in developing therapeutic vaccines encompassing various platforms to date, there remains a need to identify and provide new and more effective immunotherapeutic vaccines and related treatment regimes. Thus, the present disclosure seeks to address this and other needs.

### SUMMARY

In a first aspect, provided herein is a method comprising administering to a subject having cancer, a vaccine and an IL-2Rβ-activating amount of a long acting IL-2Rβ-biased agonist, to be described in greater detail herein.

In a second aspect, provided herein is a method of enhancing the therapeutic effectiveness of a cancer vaccine, comprising administering to a subject having cancer a therapeutic cancer vaccine and an IL-2Rβ-activating amount of a long-acting IL-2Rβ-biased agonist, wherein the long-acting IL-2Rβ-biased agonist is effective to improve the subject's response to the vaccine.

In yet a further, third aspect, provided herein is a method of treating cancer in a subject, comprising administering to a subject an IL-2Rβ-activating amount of a long-acting IL-2Rβ-biased agonist and a vaccine in an amount effective to treat cancer, wherein when evaluated in a mouse model of the cancer, treatment is effective to prolong survival over administration of the vaccine and a non-long acting version of the IL-2R agonist by at least 15 days, based upon the time delay between 50% maximum tumor growth for both treatment regimens.

In yet a fourth aspect, the disclosure provides a method of inhibiting accumulation of regulatory T cells (Tregs) in a subject undergoing treatment for cancer, comprising administering to the subject an IL-2Rβ-activating amount of a long acting IL-2Rβ-biased agonist and a vaccine in an amount effective to treat a cancerous tumor, where when evaluated in a mouse model of cancer, the treatment is effective to inhibit accumulation of regulatory T cells selected from the group consisting of CD4+ Tregs, CD25+ Tregs, and FoxP3+ Tregs in the tumor by an amount that is enhanced over that observed upon administration of a non-long acting version of the IL-2R agonist and the vaccine.

By way of clarity, with regard to the sequence of administering, the vaccine and the long acting IL-2Rβ-biased agonist may be administered concurrently or sequentially and in any order, and via the same and/or different routes of administration. Moreover, treatment may comprise a single cycle of therapy, or may comprise multiple cycles.

In one or more embodiments related to any one or more of the aspects or embodiments provided herein, the long-acting IL-2Rβ-biased agonist is administered at a dose that is less than or equal to about 0.7 mg/kg. In one or more particular embodiments, the long-acting IL-2Rβ-biased agonist is administered at a dose that is less than about 0.7 mg/kg.

In one or more embodiments related to any one or more of the foregoing aspects, the vaccine is administered to the subject separately from the long acting IL-2Rβ-biased agonist.

In yet one or more further embodiments, the vaccine is administered to the subject prior to administering the long acting IL-2Rβ-biased agonist. For example, in one or more embodiments, the vaccine and the long acting IL-2Rβ-biased agonist are both administered on day 1 of treatment. In one or more alternative embodiments, the vaccine is administered on day 1 of treatment and the long acting IL-2Rβ-biased agonist is administered at any one of days 1 to 4 of treatment. For example, the long acting IL-2Rβ-biased agonist is administered on any one of days 1, 2, 3, or 4 of treatment, or even thereafter.

In some embodiments, the subject is a human subject.

In one or more additional embodiments, the cancer is a solid cancer. For example, the cancer may be selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

In yet one or more further embodiments, the long acting IL-2Rβ-biased agonist is administered at a dose in a range of less than or equal to about 0.7 mg/kg to about 0.2 mg/kg. In yet one or more further embodiments, the long acting IL-2Rβ-biased agonist is administered at a dose in a range of less than about 0.7 mg/kg to about 0.2 mg/kg. In some further embodiments, the long-acting IL-2Rβ-biased agonist is administered at a dose that is less than or equal to about 0.7 mg/kg to about 0.3 mg/kg, or in a dose ranging from less than about or equal to about 0.7 mg/kg to about 0.5 mg/kg. Illustrative dosage amounts for the long-acting IL-2Rβ-biased agonist include, for example, 0.7 mg/kg; 0.65 mg/kg, 0.6 mg/kg, 0.5 mg/kg, 0.4 mg/kg, 0.3 mg/kg, and 0.2 mg/kg.

In some embodiments relating to any one or more of the foregoing aspects, when treating a solid cancerous tumor, the method is effective to result in a reduction in solid tumor size of at least about 25% when evaluated after 1 cycle of treatment.

In some embodiments, the long acting IL-2Rβ-biased agonist comprises aldesleukin releasably covalently attached to polyethylene glycol. In yet some additional embodiments, the long acting IL-2Rβ-biased agonist comprises aldesleukin releasably covalently attached to from 4, 5 and 6 polyethylene glycol polymers. In yet some further embodiments, the long acting IL-2Rβ-biased agonist comprises aldesleukin releasably covalently attached to an average of about 6 polyethylene glycol polymers. In one or more additional embodiments, the polyethylene glycol polymers that are releasably covalently attached to aldesleukin are branched.

In yet some further embodiments related to any one or more of the foregoing aspects, the vaccine is selected from, for example, an antigen vaccine, a whole cell vaccine, a dendritic cell vaccine, and a DNA vaccine. In one or more embodiments, the vaccine is an allogenic vaccine. Alternatively, in some embodiments, the vaccine is an autologous vaccine. In some further particular embodiments, the vaccine is an antigen vaccine. In one or more related embodiments, the antigen vaccine comprises a tumor-specific antigen. For example, in some embodiments, the tumor-specific antigen is selected from a cancer-testis antigen, a differentiation antigen, and a widely-occurring over-expressed tumor associated antigen.

In yet some further embodiments, the vaccine comprises a neoantigen.

In yet a further aspect, provided is a kit comprising an IL-2Rβ-activating amount of a long acting IL-2Rβ-biased agonist and a vaccine, accompanied by instructions for use in treating a subject having cancer.

In one or more embodiments of the kit, the long acting IL-2Rβ-biased agonist and the vaccine are comprised in a single composition for administration to the subject, where the single composition optionally comprises a pharmaceutically acceptable excipient.

In some alternative embodiments of the kit, the long acting IL-2Rβ-biased agonist and the vaccine are provided in separate containers, and the kit comprises instructions for administering the vaccine and the long-acting IL-2Rβ-biased agonist separately to the subject.

In some embodiments of the kit, both the long-acting IL-2Rβ-biased agonist and the vaccine are in solid form. In one or more related embodiments, the long acting IL-2Rβ-biased agonist and the vaccine are in a solid form suitable for reconstitution in an aqueous diluent.

In yet one or more further embodiments, each of the long acting IL-2Rβ-biased agonist and the vaccine are comprised within separate compositions each comprising a pharmaceutically acceptable excipient.

In yet some additional embodiments, both the composition comprising the long acting IL-2Rβ-biased agonist and the composition comprising the vaccine contain less than 5 percent by weight water.

Additional aspects and embodiments are set forth in the following description and claims, and the disclosure should not be considered to be limited in this regard.

### BRIEF DESCRIPTION OF THE DRAWINGS

FIGS. 1A-1H. These figures illustrate immune cell alterations in B16F10 mouse melanoma models following treatment with a single dose of RSLAIL-2 or 5 daily doses of aldesleukin as described in detail in Example 2. Tumor-infiltrating lymphocytes were isolated from animals at the time points indicated and immune cell populations were assessed by flow cytometry. Each data point represents an individual mouse tumor and the line represents the mean. Data were combined from 2 to 4 independent studies with 3 to 4 replicates at each time point. FIG. 1A shows total percentage of CD8 T cells in the tumor at various time points (days 5, 7, and 10) following treatment with each of vehicle (open circles), aldesleukin (filled squares) and RSLAIL-2 (filled triangles); FIG. 1B shows percentage of memory CD8 T cells in the tumor at various time points following treatment with each of vehicle (open circles), aldesleukin (filled squares) and RSLAIL-2 (filled triangles); FIG. 1C shows percentage of activated NK cells in the tumor at various time points (days 5, 7, and 10) following treatment with each of vehicle (open circles), aldesleukin (filled squares) and RSLAIL-2 (filled triangles); FIGS. 1D and 1E show percentage of CD4 T cells in the tumor at various time points (days 5, 7, and 10) following treatment; FIG. 1F shows percentage of CD4 Treg cells in the tumor at various time points (days 5, 7, and 10) following treatment; FIG. 1G shows percentage of Treg cells of total CD4 cells following treatment; and FIG. 1H provides the ratio of total CD8 cells to Treg cells following treatment.

FIG. 2 is a graph demonstrating tumor pharmacokinetics of RSLAIL-2 (closed squares) (and its released active conjugated-IL-2 forms, closed circles) in comparison to unmodified IL-1 (aldesleukin, closed upside down triangles) as described in Example 3.

FIGS. 3A-3H are plots showing tumor size (mm²) over the course of treatment in C57BL/6 mice bearing established subcutaneous B16 tumors, followed by vaccination with (i) a cocktail formulation containing GP-100, an illustrative peptide vaccine; an anti-CD40 mAb; and a TLR-7 agonist, R848 (Resiquimod, an imidazoquinoline); alone or (ii) in combination with a long acting IL-2Rαβ-biased agonist, RSLAIL-2 (0.2 mg/kg based on IL-2) or (iii) in combination with either high dose or low dose unmodified IL-2 (aldesleukin) as described in detail for the various treatment groups in detail in Example 4.

FIG. 4 is a graph showing average tumor size (mm²) over the course of treatment in C57BL/6 mice bearing established subcutaneous B16 tumors for each of the study groups described in detail in Example 4.

FIG. 5 is a plot associated with gp100-specific T cell function, i.e., demonstrating IFN-g+ Tcells (expressed as a percentage of pmel-1) over the course of treatment in C57BL/6 mice bearing established subcutaneous B16 tumors for each of the study groups described in detail in Example 4. The plot indicates a stable and persistent IGN-g+T cell (pmel-1) response at above 90% extending to about 40 days post vaccination for the GP100/anti-CD40/TRL-7 agonist/RSLAIL-2 treatment group; the vaccine/RSLAIL-2 combination therapy reached and maintained the highest percentage of IFN-g+ Tcell (pmel-1) response over the other treatment groups. Additionally, the vaccine/RSAIL-2 combination therapy-induced IGN-g+T cell (pmel-1) response was slower to decline than in the other treatment groups.

FIG. 6 is a plot demonstrating percent survival over the course of treatment in C57BL/6 mice bearing established subcutaneous B16 tumors for each of the study groups described in detail in Example 4. Consistent with the plots showing tumor size over the course of treatment (FIGS. 3A-3H and FIG. 4), survival for the vaccine/RSLAIL-2 treatment group (GRP8) was significantly prolonged in comparison to the other treatment groups.

FIG. 7 is a plot demonstrating percent pmel-cells (expressed as a percentage of total CD8+ T cells) over the course of treatment in C57BL/6 mice bearing established subcutaneous B16 tumors for each of the study groups described in Example 4. RSLAIL-2, when combined with the GP-100 vaccine, exhibited a notably elevated pmel-1 response when compared to both high dose and low dose IL-1 treatment coupled with peptide vaccine therapy.

FIG. 8 is a plot showing regulatory T cells, CD25+Foxp3+ T cells, expressed as a percentage of CD4 cells over the course of treatment in C57BL/6 mice bearing established subcutaneous B16 tumors for each of the study groups described in Example 4. As can be seen from the plot, the percentage of RSLAIL-2-induced regulatory T cells decreases rapidly around the end of each dosing cycle.

FIG. 9 is a bar graph indicating numbers of Thy1.1+ pmel-1 cells/gram of tumor over the course of treatment at each of days 5, 7, 10 and 30 in C57BL/6 mice bearing established subcutaneous B16 tumors for each of the study groups described in Example 5.

FIG. 10 is a bar graph indicating numbers of Thy1.1+ pmel-1 cells/gram of spleen tissue at each of days 5, 7, 10 and 30 in C57BL/6 mice bearing established subcutaneous B16 tumors for each of the study groups described in Example 5.

FIG. 11 provides the NOUS-020 insert sequence that corresponds to 20 neoantigens from the CT26 murine tumor cell line, as described in the Examples (e.g., Examples 6-9), SEQ ID NO:5.

FIGS. 12A and 12B. As described in Example 6, FIGS. 12A and 12B illustrate the immunogenicity of the illustrative mouse neoantigenic cancer vaccine, NOUS-020, for use in the murine studies described herein. Analysis of T cell responses measured 3 weeks post immunization in naive mice by IFN-γ ELISpot on single mutated peptides is shown in FIG. 12A and on a pool of 20 peptides by intracellular cytokine staining in FIG. 12B (pool of peptides). Shown are the responses to the 5 immunogenic peptides (#3, 10, 17, 18, 19). ID epitopes correspond to position of the antigen in the construct where SFC refers to Spot Forming Cells. As shown, the illustrative mouse neoantigenic cancer vaccine, NOUS-020 GAd induces CD4 and CD8 T cells.

FIG. 13A provides a schematic of constructs showing the neontigens inducing the CD8 and CD4 response in the study described in Example 7. FIG. 13B provides an analysis of T cell responses measured post GAd/MVA immunization in naive mice by IFN-γ ELISpot on pool of 20 vaccine encoded neo-antigens.

FIGS. 14A-14F are plots of CT26 tumor growth in Balb/c mice receiving either no treatment, treatment with NOUS-020 GAd vaccine alone, treatment with RSLAIL-2 alone, or treatment with a combination of NOUS-020 GAd vaccine and RSLAIL-2 as described in Example 8. FIG. 14A provides results for the control group (untreated); FIG. 14B demonstrates volume of CT26 tumors in mice treated with GAd vaccine alone; FIGS. 14C and 14D demonstrate volume of CT26 tumors in mice treated with RSLAIL-2 (administered at either day 0 or 7, respectively) and concomitant at Day 0 (FIG. 14E) or sequential administration (FIG. 14F) of RSLAIL-2 and GAd, respectively.

FIG. 15A is a plot of tumor volume in individual mice with established tumors treated with RSAIL-2 alone; FIG. 15B is a plot of tumor volume in individual mice with established tumors treated with a combination of NOUS-020 vaccine and RSLAIL-2 as described in Example 9. CR=complete response PR=partial response (>40% tumor shrinkage).

FIGS. 16A and 16B provide an analysis of immune response at day 54 measured in the spleen of mice responding to treatment with (i) RSLAIL-2 only, and (ii) NOUS-020 and RSLAIL-2, respectively, as described in Example 9. The T cell response against the pool of top 5 immunogenic neo-antigens and against the remaining 15 neoantigens encoded by the vaccine were quantified by ICS. Dashed and solid line represent a threshold for a positive response respectively for CD4 and CD8 T cells.

FIG. 17A is a graph showing average tumor size (mm²) in BALB/c mice bearing established CT26 tumors for each of the study groups described in Example 10.

FIG. 17B is a plot demonstrating percent survival over the course of treatment in BALB/c mice bearing established subcutaneous CT26 tumors for each of the study groups described in detail in Example 10. Consistent with the plots, survival for the AH1 vaccine/RSLAIL-2 treatment group was significantly prolonged in comparison to the other treatment groups.

FIG. 18A is a bar graph indicating the ratio of CD8+ T cells to Tregs in spleen tissue in BALB/c mice bearing established subcutaneous CT26 tumors and treated as described for each of the study groups in Example 10. FIG. 18B is a bar graph indicating the ratio of CD8+ T cells to Tregs in tumor tissue in BALB/c mice bearing established subcutaneous CT26 tumors and treated as described for each of the study groups in Example 10.

### DETAILED DESCRIPTION

### Terms

As used in this specification, the singular forms “a,” “an,” and “the” include plural referents unless the context clearly dictates otherwise.

In describing and claiming certain features of this disclosure, the following terminology will be used in accordance with the definitions described below unless indicated otherwise.

It is to be understood that wherever aspects are described herein with the language “comprising,” otherwise analogous aspects described in terms of “consisting of” and/or “consisting essentially of” are also provided.

“Water soluble, non-peptidic polymer” refers to a polymer that is at least 35% (by weight) soluble in water at room temperature. Preferred water soluble, non-peptidic polymers are however preferably greater than 70% (by weight), and more preferably greater than 95% (by weight) soluble in water. Typically, an unfiltered aqueous preparation of a “water-soluble” polymer transmits at least 75% of the amount of light transmitted by the same solution after filtering. Preferably, such unfiltered aqueous preparation transmits at least 95% of the amount of light transmitted by the same solution after filtering. Most preferred are water-soluble polymers that are at least 95% (by weight) soluble in water or completely soluble in water. With respect to being “non-peptidic,” a polymer is non-peptidic when it contains less than 35% (by weight) of amino acid residues.

The terms “monomer,” “monomeric subunit” and “monomeric unit” are used interchangeably herein and refer to one of the basic structural units of a polymer. In the case of a homo-polymer, a single repeating structural unit forms the polymer. In the case of a co-polymer, two or more structural units are repeated—either in a pattern or randomly—to form the polymer. Preferred polymers used in connection with the present invention are homo-polymers. The water-soluble, non-peptidic polymer comprises one or more monomers serially attached to form a chain of monomers.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present invention will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary.

“Branched,” in reference to the geometry or overall structure of a polymer, refers to a polymer having two or more polymer “arms” or “chains” extending from a branch point or central structural feature.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. PEG polymers are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

A “physiologically cleavable” or “hydrolyzable” or “degradable” bond is a relatively labile bond that reacts with water (i.e., is hydrolyzed) under physiological conditions. The tendency of a bond to hydrolyze in water may depend not only on the general type of linkage connecting two atoms within a given molecule but also on the substituents attached to these atoms. Appropriate hydrolytically unstable or weak linkages may include but are not limited to carboxylate ester, phosphate ester, anhydrides, acetals, ketals, acyloxyalkyl ether, imines, orthoesters, peptides, oligonucleotides, thioesters, and carbonates.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages may generally include but are not limited to the following: carbon-carbon bonds (e.g., in aliphatic chains), ethers, amides, amines, and the like. Generally, a stable linkage is one that exhibits a rate of hydrolysis of less than about 1-2% per day under physiological conditions. Hydrolysis rates of representative chemical bonds can be found in most standard chemistry textbooks.

A covalent “releasable” linkage, for example, in the context of a polyethylene glycol that is covalently attached to an active moiety such as interleukin-2, is one that, under physiological conditions by any suitable release mechanism, releases or detaches the polyethylene glycol polymer moiety from the active moiety such as interleukin-2.

Reference to a long acting IL-2Rαβ-biased agonist as described herein is meant to encompass pharmaceutically acceptable salt forms thereof.

“Substantially” or “essentially” means nearly totally or completely, for instance, 95% or greater of a given quantity.

Similarly, “about” or “approximately” as used herein means within plus or minus 5% of a given quantity.

“Pharmaceutically acceptable excipient” or “pharmaceutically acceptable carrier” refers to a component that may be included in the compositions described herein and causes no significant adverse toxicological effects to a subject.

The term “patient,” or “subject” as used herein refers to a living organism suffering from or prone to a condition that can be prevented or treated by administration of a compound or composition or combination as provided herein, such as a cancer, and includes both humans and animals. Subjects include, but are not limited to, mammals (e.g., murines, simians, equines, bovines, porcines, canines, felines, and the like), and preferably are human.

“Administering” refers to the introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. A therapeutic agent can also be administered via a non-parenteral route, or orally. Other non-parenteral routes include a topical, epidermal or mucosal route of administration, for example, intranasally, vaginally, rectally, sublingually or topically.

A “therapeutically effective amount” or “therapeutically effective dosage” of a therapeutic agent is any amount of the agent that, when used alone or in combination with another therapeutic agent, (i) protects a subject against the onset of a disease, or (ii) promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects, in animal model systems predictive of efficacy in humans, or by assaying the activity of the agent in in vitro assays.

By way of example for the treatment of tumors, a therapeutically effective amount of an agent or a combination of agents is an amount that inhibits cell growth or tumor growth by at least about 10%, by at least about 20%, by at least about 30%, by at least about 40%, by at least about 50%, by at least about 60%, by at least about 70%, or by at least about 80%, by at least about 90%, at least about 95%, or at least about 100% relative to untreated subjects. Preferably, a therapeutically effective amount is an amount that inhibits cell growth or tumor growth by at least about 30%.

### Overview

In an effort to address at least some of the shortcomings associated with current anti-cancer vaccine strategies, such as for example, weak immune responses, provided herein is a method of comprising administering to a subject having cancer, a vaccine and an IL-2Rβ-activating amount of a long acting IL-2Rβ-biased agonist. While cytokines such as IL-2, as well as other adjuvants, have been explored to improve anti-tumor responses to cancer vaccines, further enhancements are needed to provide durable, reproducible and effective vaccine-based cancer therapies. Thus, the present disclosure is based, at least in part, on the discovery of a particularly beneficial therapeutic combination comprising a cancer vaccine and a long-acting IL-2R agonist, and more specifically, an IL-2Rβ-biased agonist.

Il-2 stimulates immune cell proliferation and activation through a receptor-signaling complex containing alpha (IL2Rα, CD25), beta (IL2Rβ, CD122) and common gamma chain receptors (γ_{c}′ CD132). At high doses, IL2 binds to heterodimeric IL2Rβγ receptor leading to desired expansion of tumor killing CD8+ memory effector T (CD8 T) cells. However, IL2 also binds to its heterotrimeric receptor IL2Rαβγ with greater affinity, which expands immuosuppressive CD4+, CD25+ regulatory T cells (Tregs), which may lead to an undesirable effect for cancer immunotherapy. Thus, in an effort to overcome one or more drawbacks associated with IL-2-enhanced anti-cancer vaccination strategies, provided herein is a treatment modality that combines therapeutic cancer vaccination with administration of an IL-2Rαβ-biased agonist, and in particular, a long acting IL-2Rαβ-biased agonist. Without being bound by theory, the Applicants have discovered that by utilizing a long-acting IL-2 compound in which a region that interacts with the IL2Rα subunit responsible for activating immunosuppressive Tregs is masked (i.e., its activity suppressed or dampened), i.e., a long acting IL-2Rαβ-biased agonist, one can selectively expand vaccination-induced T-cell responses to achieve superior therapeutic efficacy, as will become apparent from the instant disclosure and supporting examples.

### Vaccines

The treatment methods provided herein comprise administering a vaccine, i.e., for stimulating a cancer specific-immune response, e.g., innate and adaptive immune responses, for generating host immunity against a cancer. The compositions and methods provided herein find use in, among other things, both clinical and research applications. Various cancer immunogens can be administered in accordance with the methods described herein, and the invention is not limited in this regard. It is the Applicant's view that successful vaccination outcomes can be achieved via the IL-2 pathway (i.e., via co-administration, along with a vaccine, of a long acting IL-2Rαβ-biased agonist) to simulate the desired T-cell responses due to the complementary nature of cancer vaccines and a long acting IL-2Rαβ-biased agonist. That is to say, administration of a long acting IL-2Rαβ-biased agonist in combination with vaccination can be employed to achieve any one or more of the following: (i) greatly enhance the efficacy and utility of multiple classes of vaccines, (ii) promote a strong T-cell response, and (iii) increase immune activity against high, medium and low affinity antigens. The supporting examples illustrate the utility of this approach. More particularly, as illustrated herein, a long acting IL-2Rαβ-biased agonist, i.e., RSLAIL-2, in combination with vaccination to stimulate a cancer-specific immune response, is effective to provide one or more of the following: a significantly enhanced anti-tumor effect, improved survival, and expanded proliferation of pmel-1 CD8+ T cells in tumor tissue over either vaccination or the long acting IL-2Rαβ-biased agonist when administered singly (i.e., alone).

Illustrative vaccines include, but are not limited to, for example, antigen vaccines, whole cell vaccines, dendritic cell vaccines, and DNA vaccines. Moreover, depending upon the particular type of vaccine, the vaccine composition may include one or more suitable adjuvants known to enhance a subject's immune response to the vaccine. The vaccine may, for example, be cellular-based, i.e., created using cells from the patient's own cancer cells to identify and obtain an antigen. Exemplary vaccines include tumor-cell based and dendritic-cell based vaccines, where activated immune cells from the subject are delivered back to the same subject, along with other proteins, to further facilitate immune activation of these tumor antigen primed immune cells. Tumor cell based vaccines include whole tumor cells and gene-modified tumor cells. Whole tumor cell vaccines may optionally be processed to enhance antigen presentation, e.g., by irradiation of either the tumor cells or tumor lysates). Vaccine administration may also be accompanied by adjuvants such as bacillus calmette-guerin (BCG) or keyhole limpet hemocyanin (KLH), depending upon the type of vaccine employed. Plasmid DNA vaccines may also be used, and can be administered via direct injection or biolistically. Also contemplated for use are peptide vaccines, viral gene transfer vector vaccines, and antigen-modified dentritic cells (DCs).

In some embodiments, the vaccine is a therapeutic cancer peptide-based vaccine. Peptide vaccines can be created using known sequences or from isolated antigens from a subject's own tumor(s), and include neoantigens and modified antigens. Illustrative antigen-based vaccines include those where the antigen is a tumor-specific antigen. For example, the tumor-specific antigen may be selected from a cancer-testis antigen, a differentiation antigen, and a widely-occurring over-expressed tumor associated antigen, among others. Recombinant peptide vaccines, based on peptides from tumor-associated antigens, when used in the instant method, may be administered or formulated with, an adjuvant or immune modulator. Illustrative antigens for use in a peptide-based vaccine include, but are not limited to, the following, since this list is meant to be purely illustrative. For example, a peptide vaccine may comprise a cancer-testis antigen such as MAGE, BAGE, NY-ESO-1 and SSX-2, encoded by genes that are normally silenced in adult tissues but transcriptionally reactivated in tumor cells. Alternatively, the peptide vaccine may comprise a tissue differentiation associated antigen, i.e., an antigen of normal tissue origin and shared by both normal and tumorous tissue. For example, the vaccine may comprise a melanoma-associated antigen such as gp100, Melan-A/Mart-1, MAGE-3, or tyrosinase; or may comprise a prostate cancer antigen such as PSA or PAP. The vaccine may comprise a breast cancer-associated antigen such as mammaglobin-A. Other tumor antigens that may be comprised in a vaccine for use in the instant method include, for example, CEA, MUC-1, HER1/Nue, hTERT, ras, and B-raf. Other suitable antigens that may be used in a vaccine include SOX-2 and OCT-4, associated with cancer stem cells or the EMT process.

Antigen vaccines include multi-antigen and single antigen vaccines. Exemplary cancer antigens may include peptides having from about 5 to about 30 amino acids, or from about 6 to 25 amino acids, or from about 8 to 20 amino acids.

As described above, an immunostimulatory adjuvant (different from RSLAIL-2) may be used in a vaccine, in particular a tumor-associated antigen based vaccine, to assist in generating an effective immune response. For example, a vaccine may incorporate a pathogen-associated molecular pattern (PAMP) to assist in improving immunity. Additional suitable adjuvants include monophosphoryl lipid A, or other lipopolysaccharides; toll-like receptor (TLR) agonists such as, for example, imiquimod, resiquimod (R-848), TLR3, IMO-8400, and rintatolimod. Additional adjuvants suitable for use include heat shock proteins.

Also suitable for use in the methods provided herein are genetic vaccines. A genetic vaccine typically uses viral or plasmid DNA vectors carrying expression cassettes. Upon administration, they transfect somatic cells or dendritic cells as part of the inflammatory response to thereby result in cross-priming or direct antigen presentation. In some embodiments, a genetic vaccine is one that provides delivery of multiple antigens in one immunization. Genetic vaccines include DNA vaccines, RNA vaccines and viral-based vaccines.

DNA vaccines for use in the instant methods are bacterial plasmids that are constructed to deliver and express tumor antigen. DNA vaccines may be administered by any suitable mode of administration, e.g., subcantaneous or intradermal injection, but may also be injected directly into the lymph nodes. Additional modes of delivery include, for example, gene gun, electroporation, ultrasound, laser, liposomes, microparticles and nanoparticles.

More particularly, in some embodiments, the vaccine comprises a neoantigen, or multiple neoantigens. That is to say, in some embodiments, the vaccine is a neoantigen-based vaccine. This approach is exemplified in Examples 6-9 herein using a murine cancer model. For example, in some embodiments, a neoantigen-based vaccine (NBV) composition may encode multiple cancer neoantigens in tandem, where each neoantigen is a polypeptide fragment derived from a protein mutated in cancer cells. For instance, a neoantigenic vaccine may comprise a first vector comprising a nucleic acid construct encoding multiple immunogenic polypeptide fragments, each of a protein mutated in cancer cells, where each immunogenic polypeptide fragment comprises one or more mutated amino acids flanked by a variable number of wild type amino acids from the original protein, and each polypeptide fragment is joined head-to-tail to form an immunogenic polypeptide. The lengths of each of the immunogenic polypeptide fragments forming the immunogenic polypeptide can vary.

Viral gene transfer vector vaccines may also be used; in such vaccines, recombinant engineered virus, yeast, bacteria or the like is used to introduce cancer-specific proteins to the patient's immune cells. In a vector-based approach, which can be tumor lytic or non-tumor lytic, the vector can increase the efficiency of the vaccine due to, for example, its inherent immunostimulatory properties. Illustrative viral-based vectors include those from the poxviridae family, such as vaccinia, modified vaccinia strain Ankara and avipoxviruses. Also suitable for use is the cancer vaccine, PROSTVAC, containing a replication-competent vaccinia priming vector and a replication-incompetent fowlbox-boosting vector. Each vector contains transgenes for PSA and three co-stimulatory molecules, CD80, CD54 and CD58, collectively referred to as TRICOM. Other suitable vector-based cancer vaccines include Trovax and TG4010 (encoding MUC1 antigen and IL-2).

Additional vaccines for use include bacteria and yeast-based vaccines such as recombinant Listeria monocytogenes and Saccharomyces cerevisae.

As described previously, the foregoing vaccines may be combined and/or formulated with adjuvants and other immune boosters to increase efficacy. Moreover, depending upon the particular vaccine, administration may be either intratumoral or non-intratumoral (i.e., systemic). In some embodiments, the vaccine that is administered with a long acting IL-2Rβ-biased agonist is not a glycoprotein 100 (GP100) vaccine. In some other embodiments, the vaccine is not a gp100 vaccine that is administered as a component of a formulation cocktail comprising an anti-CD-40 agonist and a TLR7 agonist.

The cancer vaccine may be administered by any suitable administration route as described herein, for example, intradermal, intravenous, subcutaneous, intranodel, intralymphatic, intratumoral, and the like.

### Long Acting, IL-2Rβ-Biased Agonist

The methods, formulations, kits and the like described herein involve the administration of a long acting, IL-2Rβ-biased agonist. In this regard, the disclosure is not limited to any particular long acting, IL-2Rβ-biased agonist so long as the agonist exhibits an in vitro binding affinity for IL-2Rβ that is at least 5 times greater (more preferably at least 10 times greater) than the binding affinity for IL-2Rαβ in the same in vitro model, and has at least an effective 10-fold in vivo half-life greater than IL-2 (half-life based on the in-vivo disappearance of IL-2). By way of example, it is possible to measure binding affinities against IL-2 as a standard. In this regard, the RSLAIL-2 referenced in Example 1 herein exhibits about a 60-fold decrease in affinity to IL-2Rαβ relative to IL-2, but only about a 5-fold decrease in affinity IL-2Rβ relative to IL-2.

Non-limiting examples of long acting, IL-2Rβ-biased agonists are described in International Patent Publication Nos. WO 2012/065086 and in WO 2015/125159. An exemplary long acting, IL-2Rβ-biased agonist is RSLAIL-2 referenced in Example 1 in the present application, where the releasable PEG is based upon a 2,7,9-substituted fluorene as shown below, with poly(ethylene glycol) chains extending from the 2- and 7-positions on the fluorene ring via amide linkages (fluorene-C(O)—NH˜), and releasable covalent attachment to IL-2 via attachment to a carbamate nitrogen atom attached via a methylene group (—CH₂—) to the 9-position of the fluorene ring. In this regard, RSLAIL-2 is a composition comprising compounds encompassed by the following formula:

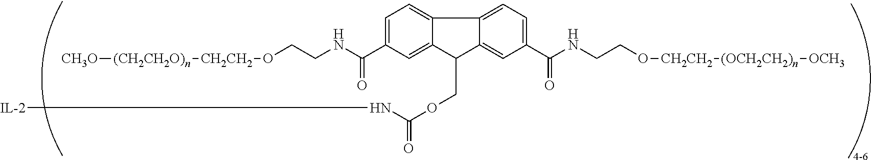

wherein IL-2 is a residue of IL-2, and pharmaceutically acceptable salts thereof, where “n” is an integer from about 3 to about 4000. As indicated in the formula above, the IL-2 molecule preferably possesses 4, 5, or 6 branched polyethylene glycol moieties as shown above covalently attached thereto. In one or more embodiments, the composition contains no more than 10% (based on a molar amount), and preferably no more than 5% (based on a molar amount), of compounds encompassed by the following formula

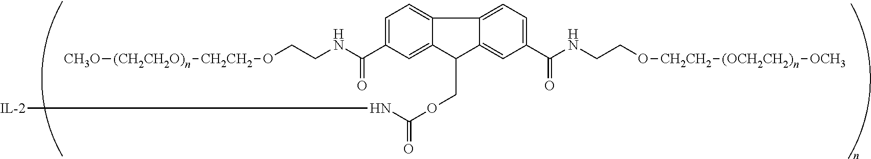

wherein IL-2 is a residue of IL-2, (n) (referring to the number of polyethylene glycol moieties attached to IL-2) is an integer selected from the group consisting of 1, 2, 3, 7 and >7, and pharmaceutically acceptable salts thereof. In some embodiments, RSLAIL-2 possesses on average about six polyethylene glycol moieties attached to IL-2.

To determine average degree of PEGylation for a composition such as the RSLAIL-2 composition described herein, typically the protein is quantified by a method such as an bicinchoninic acid (BCA) assay or by UV analysis, to determine moles of protein in the sample. The PEG moieties are then released by exposing the sample to conditions in which the PEG moieties are released, and the released PEG is then quantified (e.g., by BCA or UV) and correlated with moles protein to determine average degree of PEGylation.

In some further embodiments, RSLAIL-2 is generally considered to be an inactive prodrug, i.e., inactive upon administration, and by virtue of slow release of the polyethylene glycol moieties in vivo, providing active conjugated forms of interleukin-2, effective to achieve sustained concentrations at the tumor site. As provided in Example 2, RSLAIL-2 may be considered to be a CD-122 (also known as IL-2R(3) agonist, that is, a molecule capable of activating or stimulating CD-122 (IL-2Rβ). Moreover, RSLAIL-2 may be considered to be a CD-122 agonist that selectively binds and activates IL-2Rβγ over IL-2Rαβγ.

Additional exemplary compositions of RSLAIL-2 comprise compounds in accordance with the above formula wherein the overall polymer portion of the molecule has a weight average molecular weight in a range of from about 250 Daltons to about 90,000 Daltons. Additional suitable ranges include weight average molecular weights in a range selected from about 1,000 Daltons to about 60,000 Daltons, in a range of from about 5,000 Daltons to about 60,000 Daltons, in a range of about 10,000 Daltons to about 55,000 Daltons, in a range of from about 15,000 Daltons to about 50,000 Daltons, and in a range of from about 20,000 Daltons to about 50,000 Daltons.

Additional illustrative weight-average molecular weights for the polyethylene glycol polymer portion include about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. In some embodiments, the weight-average molecular weight of the polyethylene glycol polymer is about 20,000 daltons.

As described above, the long-acting, IL-2Rβ-biased agonist may be in the form of a pharmaceutically-acceptable salt. Typically, such salts are formed by reaction with a pharmaceutically-acceptable acid or an acid equivalent. The term “pharmaceutically-acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-2 as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In reference to the foregoing IL-2Rβ-biased agonist, the term “IL-2” as used herein, refers to a moiety having human IL-2 activity. The term, ‘residue’, in the context of residue of IL-2, means the portion of the IL-2 molecule that remains following covalent attachment to a polymer such as a polyethylene glycol, at one or more covalent attachment sites, as shown in the formula above. It will be understood that when the unmodified IL-2 is attached to a polymer such as polyethylene glycol, the IL-2 is slightly altered due to the presence of one or more covalent bonds associated with linkage to the polymer(s). This slightly altered form of the IL-2 attached to another molecule is referred to a “residue” of the IL-2.

For example, proteins having an amino acid sequence corresponding to any one of SEQ ID NOs: 1 through 4 described in International Patent Publication No. WO 2012/065086 are exemplary IL-2 proteins, as are any proteins or polypeptides substantially homologous thereto. These sequences are also provided herein. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology, equivalent biological activity (although not necessarily equivalent strength of biological activity), and equivalent expression characteristics are considered substantially homologous. For purposes of determining homology, truncation of the mature sequence should be disregarded. As used herein, the term “IL-2” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties. In addition, the IL-2 can be derived from human sources, animal sources, and plant sources. One exemplary and preferred IL-2 is recombinant IL-2 referred to as aldesleukin (SEQ ID NO:3).

Conventional approaches, such as those involving radiolabeling a compound, administering it in vivo, and determining its clearance, can be used to determine whether a compound proposed to be a long-acting IL-2Rβ biased agonist is “long-acting”. For the purposes herein, the long acting nature of an IL-2Rβ biased agonist is typically determined using flow cytometry to measure STATS phosphorylation in lymphocytes at various time points after administration of the agonist to be evaluated in mice. As a reference, the signal is lost by around 24 hours with IL-2, but is sustained for a period greater than that for a long-acting IL-2Rβ-biased agonist. As an illustration, the signal is sustained over several days for the RSLAIL-2 compositions.

Considering now the IL-2Rβ bias of a long-acting agonist as described herein, Example 2 provides both in-vitro and in-vivo data related to receptor bias for exemplary compositions of RSLAIL-2. As described in Example 2, in a murine melanoma tumor model, the ratio of CD8/regulatory T cells for RSLAIL-2 when compared to IL-2 supports preferential activation of the IL-2 receptor beta over IL2 receptor alpha. Exemplary long-acting IL-2Rβ biased agonists such as RSLAIL-2 are, for example, effective to preferentially activate and expand effector CD8+ T- and NK-cells over Tregs.

Moreover, representative long-acting IL-2Rβ-biased agonists such as RSLAIL-2 provide increased tumor exposure, and preferably significantly enhanced tumor exposure relative to IL-2, for example, at least a 50-fold increased exposure, or at least a 100-fold increased exposure, or at least a 200-fold increased exposure, or at least a 300-fold increased exposure, or at least a 400-fold increased exposure, or at least a 500-fold increased exposure when normalized for equivalents of IL-2. As an illustration, the antitumor activity of RSLAIL-2 in a mouse melanoma tumor model is described in Example 3. As described therein, RSLAIL-2 was found to provide significantly enhanced tumor exposure, e.g., 500-fold, relative to IL-2 (normalized based upon IL-2 equivalents).

Based upon at least one or more of the features of a long-acting IL-2Rβ-biased agonist as described herein, provided herein are methods effective to selectively expand vaccination-induced T-cell responses in cancer patients by administering a long-acting IL-2 compound in which a region that interacts with the IL2Rα subunit responsible for activating immunosuppressive Tregs is masked, to thereby achieve superior therapeutic efficacy.

In accordance with the methods, compositions, and kits described herein, the long-acting, IL-2Rβ-biased agonist is provided in an IL-2Rβ-activating amount. One of ordinary skill in the art can determine how much of a given long-acting, IL-2Rβ-biased agonist is sufficient to provide clinically relevant agonistic activity at IL-2Rβ. For example, one of ordinary skill in the art can refer to the literature and/or administer a series of increasing amounts of the long-acting, IL-2Rβ-biased agonist and determine which amount or amounts provide clinically effective agonistic activity of IL-2Rβ. Alternatively, an activating amount of the long acting IL-2Rβ-biased agonist can be determined using the in vivo STATS phosphorylation assay described above (determined in vivo following administration) where an amount sufficient to induce STATS phosphorylation in greater than 10% of NK cells at peak is considered to be an activating amount.

In one or more instances, however, the IL-2Rβ-activating amount is an amount encompassed by one or more of the following ranges expressed in amount of protein: from about 0.01 to 100 mg/kg; from about 0.01 mg/kg to about 75 mg/kg; from about 0.02 mg/kg to about 60 mg/kg; from about 0.03 mg/kg to about 50 mg/kg; from about 0.05 mg/kg to about 40 mg/kg; from about 0.05 mg/kg to about 30 mg/kg; from about 0.05 mg/kg to about 25 mg/kg; from about 0.05 mg/kg to about 15 mg/kg; from about 0.05 mg/kg to about 10 mg/kg; from about 0.05 mg/kg to about 5 mg/kg; from about 0.05 mg/kg to about 1 mg/kg. In some embodiments, the long acting IL-2Rβ-biased agonist is administered at a dose that is less than or equal to 0.7 mg/kg. Particular illustrative dosing ranges include for example, from about 0.1 mg/kg to about 10 mg/kg, or from about 0.2 mg/kg to about 7 mg/kg or from about 0.2 mg/kg to less than about 0.7 mg/kg.

For confirmation, with respect to the long-acting, IL-2Rβ-biased agonist, the amount and extent of the activation can vary widely and still be effective when coupled with administration of a therapeutic cancer vaccine. That is to say, an amount of a long-acting, IL-2Rβ-biased agonist that exhibits only minimal agonist activity at IL-2Rβ for a sufficiently extended period of time can still be a long-acting, IL-2Rβ-biased agonist so long as when administered with a cancer vaccine, the methods, compositions, and kits described herein enable a clinically meaningful response. In some instances, due to (for example) synergistic interactions and responses, only minimal agonist activity of IL-2Rβ may be required when accompanied by anti-cancer vaccination.

The treatment methods described herein can continue for as long as the clinician overseeing the patient's care deems the treatment method to be effective. Non-limiting parameters that indicate the treatment method is effective include any one or more of the following: tumor shrinkage (in terms of weight and/or volume); a decrease in the number of individual tumor colonies; tumor elimination; and progression-free survival. Change in tumor size may be determined by any suitable method such as imaging. Various diagnostic imaging modalities can be employed, such as computed tomography (CT scan), dual energy CDT, positron emission tomography and MRI.

The actual doses of the vaccine and the long-acting, IL-2Rβ-biased agonist, as well as the dosing regimen associated with the methods, compositions, and kits described herein will vary depending upon the age, weight, and general condition of the subject as well as the type and progression of the cancer being treated, the judgment of the health care professional, and the particular vaccine and long-acting, IL-2Rβ-biased agonist to be administered.

With regard to the frequency and schedule of administering the vaccine and the long acting, IL-2Rβ-biased agonist, one of ordinary skill in the art will be able to determine an appropriate frequency. For example, in a treatment cycle, a clinician can decide to administer the vaccine, either as a single dose or in a series of doses, e.g., over the course of several days or weeks). The long acting, IL-2Rβ-biased agonist is administered, either concurrently with the vaccine, or prior to vaccination, or following administration of the cancer vaccine. For example, in some treatment modalities, the long acting, IL-2Rβ-biased agonist is administered within 7 days of vaccine administration (e.g., on any one of days 1, 2, 3, 4, 5, 6, or 7). In some instances, the long acting, IL-2Rβ-biased agonist is administered within 4 days of vaccination, e.g., on any one of days 1, 2, 3, or 4. Based upon the long acting nature of the IL-2Rβ-biased agonist, such compound is typically administered relatively infrequently (e.g., once every three weeks, once every two weeks, once every 8-10 days, once every week, etc.).

Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

The treatment methods described herein are typically continued for as long as the clinician overseeing the patient's care deems the treatment method to be effective, i.e., that the patient is responding to treatment. Non-limiting parameters that indicate the treatment method is effective may include one or more of the following: tumor shrinkage (in terms of weight and/or volume and/or visual appearance); a decrease in the number of individual tumor colonies; tumor elimination; progression-free survival; appropriate response by a suitable tumor marker (if applicable), increased number of NK (natural killer) cells, increased number of T cells, increased number of memory T cells, increased number of central memory T cells, reduced numbers of regulatory T cells such as CD4+ Tregs, CD25+ Tregs, and FoxP3+ Tregs.

The methods provided herein are useful for (among other things) treating a patient suffering from cancer. For example, patients may be responsive to the vaccine alone, as well as the combination with a long acting, IL-2Rβ-biased agonist but are more responsive to the combination. By way of further example, patients may be non-responsive to either the vaccine or the long acting, IL-2Rβ-biased agonist, but are responsive to the combination. By way of still further example, patients may be non-responsive to either of the vaccine or the long acting, IL-2Rβ-biased agonist alone, but are responsive to the combination.

Administration, e.g., of the vaccine and/or the long acting, IL-2Rβ-biased agonist is typically via injection. Other modes of administration are also contemplated, such as pulmonary, nasal, buccal, rectal, sublingual and transdermal. As used herein, the term “parenteral” includes subcutaneous, intravenous, intra-arterial, intratumoral, intralymphatic, intraperitoneal, intracardiac, intrathecal, and intramuscular injection, as well as infusion injections. As described previously, the vaccine and the long acting, IL-2Rβ-biased agonist can be administered separately. Alternatively, if administration of the vaccine and the long acting, IL-2Rβ-biased agonist, is desired to be simultaneous, either as an initial dose or throughout the course of treatment or at various stages of the dosing regimen—and the vaccine and the long acting, IL-2Rβ-biased agonist are compatible together and in a given formulation—then the simultaneous administration can be achieved via administration of single dosage form/formulation (e.g., intravenous administration of an intravenous formulation that contains both immunological components). One of ordinary skill in the art can determine through routing testing whether two such components are compatible together and in a given formulation. For example, administration to a patient can be achieved through injection of a composition comprising an IL-2Rβ-biased agonist and a diluent. In addition, administration to a patient can be achieved through injection of a cancer vaccine and a diluent. Further, administration can be achieved through injection of a composition comprising both an IL-2Rβb-biased agonist, a vaccine, and a diluent. With respect to possible diluents, the diluent can be selected from the group consisting of bacteriostatic water for injection, dextrose 5% in water, phosphate-buffered saline, Ringer's solution, lactated Ringer's solution, saline, sterile water, deionized water, and combinations thereof. One of ordinary skill in the art can determine through routing testing whether two given pharmacological components are compatible together in a given formulation.

The therapeutic combination described herein, i.e., the long acting IL-2Rβ-biased agonist and vaccine, may be provided in the form of a kit. As described above, the components may be comprised in a single composition, optionally accompanied by one or more pharmaceutically acceptable excipients, or may be provided in separate containers, where the kit typically includes instructions for use. Suitable pharmaceutically acceptable excipients include those described, for example, in the Handbook of Pharmaceutical Excipients, 7^{th} ed., Rowe, R. C., Ed., Pharmaceutical Press, 2012. The kit components, e.g., compositions comprising the vaccine and the long acting IL-2Rβ-biased agonist, may be in either liquid or in solid form. In certain preferred embodiments, both the vaccine and the long acting IL-2Rβ-biased agonist are in solid form. Preferred solid forms are those that are solid dry forms, e.g., containing less than 5 percent by weight water, or preferably less than 2 percent by weight water. The solid forms are generally suitable for reconstitution in an aqueous diluent.

The presently described methods, kits and related compositions can be used to treat a patient suffering from any condition that can be remedied or prevented by the methods provided herein, such as cancer. A cancer refers a broad group of various diseases characterized by the uncontrolled growth of abnormal cells in the body, where a cancer or cancer tissue can include a tumor. Exemplary conditions are cancers, such as, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, brain cancer, breast cancer, ovarian cancer, prostate cancer, squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, Hodgkin lymphoma, non-Hodgkin lymphoma, testicular cancer, lung cancer, small cell lung cancer, brain cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, multiple myeloma, neuroblastoma, retinoblastoma and leukemias. In some particular embodiments, the cancer to be treated is a solid cancer, such as for example, breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

The present methods, kits and compositions are useful for enhancing the therapeutic effectiveness of a cancer vaccine, for example, by improving the subject's response to the vaccine. An enhanced response may be evaluated at any suitable time point during treatment, after a single round of treatment, after 2-3 cycles of treatment, etc., and by any of a number of suitable methods, including shrinkage of a tumor (partial response), i.e., an evaluation of tumor size or volume, disappearance of a tumor, a reduction in disease progression (cancer has not progressed), and analysis of one or more tumor test markers if appropriate. In some instances, an indication of efficacy of treatment can be measured in terms of a time delay between 50% maximum tumor growth when comparing treatment with a vaccine and a long-acting IL-2Rβ-biased agonist to treatment with the vaccine administered with a corresponding non-long acting version of IL-2 (e.g., dosed to achieve a comparable number of IL-2 equivalents). The comparison may be conducted in a human patient, or in a suitable animal model such as a suitable murine model of cancer. Particularly effective treatments will prolong survival, when evaluated at 50% maximum tumor growth), by at least 5 days, or at least 10 days, or at least 12 days, or at least 15 days, or by at least 20 days, or by at least 30 days or more.

The methods, kits, compositions and the like provided herein are also useful for reducing tumor growth or size (or volume) in a subject undergoing treatment. Treatment by administering a therapeutically effective amount of cancer vaccine and a long-acting IL-2Rβ-biased agonist such as provided herein to a subject having established tumors is effective, in one or more embodiments, to reduce tumor growth or size in the subject. For example, in some embodiments, one or more cycles of treatment is effective to reduce tumor size by about 25%, or by about 30%, or by about 40%, or by about 50%, or even by about 60%, or by about 70% or more when compared to the size of the tumor prior to treatment.

In yet some further embodiments, the methods, kits, compositions and the like provided herein are effective to inhibit accumulation of regulatory T cells (Tregs) in a subject undergoing treatment for cancer. In some embodiments, the method is effective, for example, when evaluated in a cancer mouse model of the corresponding cancer, to inhibit accumulation of regulatory T cells selected from the group consisting of CD4+ Tregs, CD25+ Tregs, and FoxP3+ Tregs in the tumor (i.e., any one or more of the foregoing cell types) by an amount that is enhanced over that observed upon administration of a non-long acting IL-2Rβ-biased agonist such as IL-2 and the vaccine. For example, the subject Tregs (measured either singly or as any one of the possible combinations of Tregs) may be inhibited by 1.5-fold or more, or 2-fold or more, or 3-fold or more, or even 4-fold or more, when compared to treatment with the vaccine and IL-2. The treatment may, in some embodiments, be effective to inhibit accumulation of regulatory T cells (Tregs) in a subject by at least 2-fold or more, or 3-fold or more, or even 4-fold or more, or 5-fold or more, or 6-fold or more when compared to an untreated subject.

In yet some further embodiments, the methods, kits, composition and the like provided herein are effective to stimulate Tcell and/or NK cell activity and/or proliferation in a subject. In some embodiments, the method is effective, for example, when evaluated in a cancer mouse model of the corresponding cancer, for increasing the number of CD8+ Tcells in the subject. In yet some other embodiments, the method is effective, for example, when evaluated in a cancer mouse model of the corresponding cancer, to increase the number of NK cells in the subject. For example, the subject's CD8+ T cells may be increased by 1.5-fold or more, or 2-fold or more, or 3-fold or more, or even 4-fold or more, when compared to treatment with the vaccine and unmodified IL-2. The treatment may, in some embodiments, be effective to increase the subject's CD8+ Tcells by at least 2-fold or more, or 3-fold or more, or even 4-fold or more, or 5-fold or more, or 6-fold or more when compared to an untreated subject. Similarly, the subject's NK cells may be increased by 1.5-fold or more, or 2-fold or more, or 3-fold or more, or even 4-fold or more, when compared to treatment with the vaccine and unmodified IL-2. The treatment may, in some embodiments, be effective to increase the subject's NK cell count by at least 2-fold or more, or 3-fold or more, or even 4-fold or more, or 5-fold or more, or 6-fold or more when compared to an untreated subject.

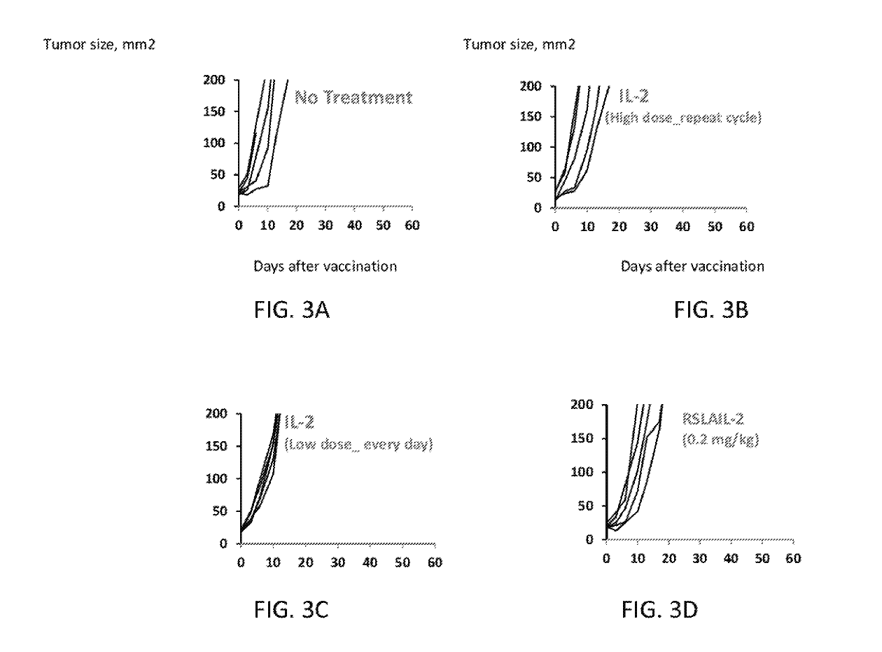

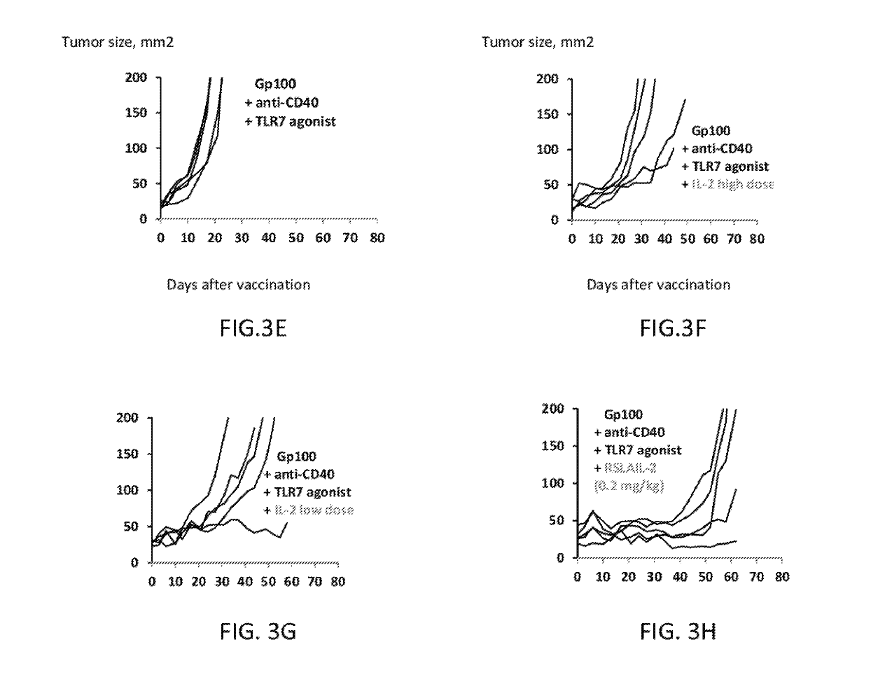

In turning to the Examples, at least Examples 4 and 5 provide further indication of the synergy arising from the administration of an illustrative therapeutic vaccine accompanied by administration of an exemplary long-acting IL-2Rβ-biased agonist such as RSLAIL-2. For example, in considering the results in FIGS. 3A-3H and FIG. 4, it can be seen that RSLAIL-2, an illustrative long acting IL-2Rαβ-biased agonist, when administered following vaccination, was effective to significantly delay tumor growth in the mouse model employed and to thereby achieve a markedly improved response when compared to vaccination alone or vaccination accompanied by administration of either high dose IL-2 or low dose IL-2. Turning to FIG. 4, it can be seen that, for example, after approximately 38 days of treatment, the average tumor size in the vaccination/RSLAIL-2 treatment group was approximately 25 mm², while the average tumor size in the closest treatment group (in terms of effectiveness in slowing tumor growth), vaccination/IL-2 low dose, was approximately 125 mm², an approximate 5-fold difference. These results highlight the superior ability of an IL-2Rαβ-biased agonist such as RSLAIL-2, when accompanying vaccine therapy, to improve the therapeutic response.

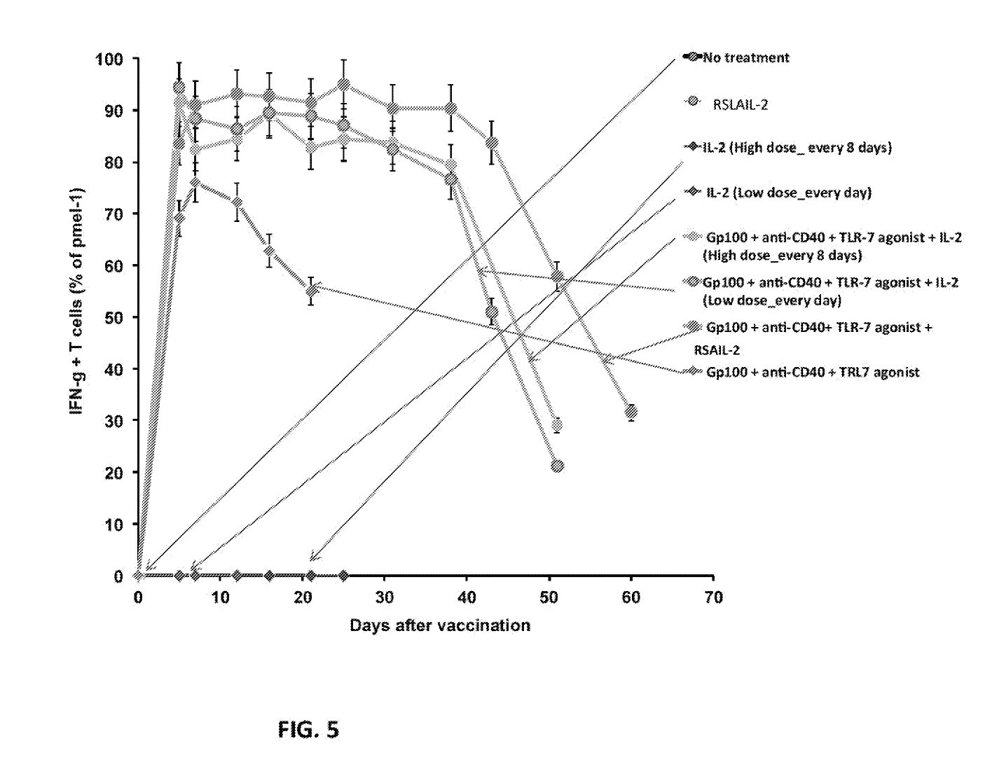

Further demonstrating the notable therapeutic results for anti-cancer vaccination accompanied by administration of the illustrative IL-2Rαβ-biased agonist, RSLAIL-2, FIG. 6 provides a plot of percent survival for each of the various treatment groups. Most significantly, 100% of subjects in the peptide vaccine/long acting IL-2Rαβ-biased agonist treatment group survived to about 57 days, with 50% survival at approximately 62 days; for the next closest treatment group in terms of positive response to therapy, i.e., the peptide vaccine/low dose IL-2 treatment group, 100% survival was observed to approximately 32 days, with 50% survival at about 48 days—an increase of approximately 15 days.

In turning to the immunostimulatory or immunodampening effects of the subject treatment methods as described in Examples 4 and 5, it can be seen that RSLAIL-2 is effective to induce a significantly higher and stable Pmel-1 response in tumor tissue than is IL-2. Moreover, when comparing vaccine treatment accompanied by administration of either high dose IL-2 or RSLAIL-2, in a fashion similar to the tumor microenvironment, RSLAIL-2 is effective to induce a significantly higher and stable Pmel-1 response in spleen than is IL-2. RSLAIL-2 effectively mediated reduction of regulatory Tcells (Tregs) at day 7 and maintained minimal numbers of Tregs in the tumor extending until at least day 30. Based upon evaluation of various immune cell types over the course of treatment (Tregs and non-Tregs), the peptide vaccine when combined with RSLAIL-2, but not with IL-2, produced higher Pmel to Tregs ratio in the tumor as well as in the spleen. Based upon at least these data, it appears that the exemplary IL-2Rαβ-biased agonist, RSLAIL-2, is markedly better than IL-2 in stably maintaining high numbers of Pmel-1 cells and low Tregs in tumor tissue and over a longer period of time. Further, RSLAIL-2 specifically inhibits accumulation of Tregs to the tumor, and promotes maintenance of a high ratio of Pmel to Tregs in tumor tissue up to day 30 of treatment.

Examples 6-9 illustrate, among other things, that when compared to administration of a neoantigen-based vaccine composition alone, a combination with RSLAIL-2 is effective to provide an immune response against a larger number of vaccine-encoded neoantigens as well as increased numbers of CD4 and CD8 T cells reactive with the vaccine-encoded neoantigens. Moreover, tumors in mice treated with the combination described herein were highly enriched in T-cells reactive to vaccine-encoded neoantigens. That is to say, the combination of a representative neoantigen-based vaccine with RSLAIL-2 led to a significant anti-tumor effect, and induced a strong neoantigen-specific immune response.

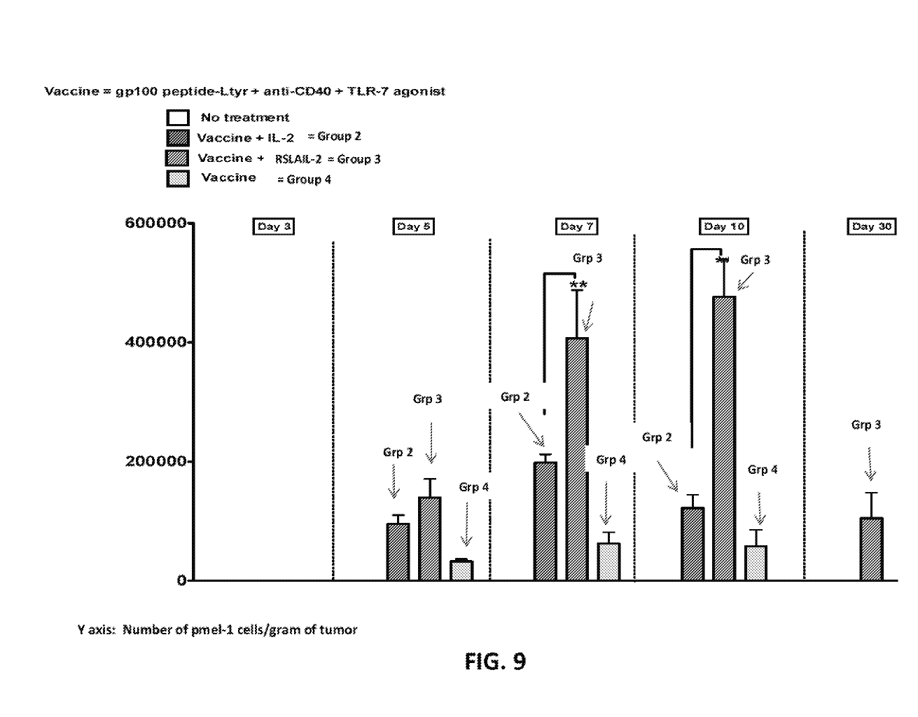

As shown in FIG. 10, the illustrative AH-1 single antigen peptide vaccine, when administered in combination with RSLAIL-2, notably delayed tumor growth and improved survival when compared to each of the AH-1 vaccine and RSLAIL-2, when administered alone. Thus, RSLAIL-2 when combined with a peptide-based cancer vaccine, was effective to delay tumor growth and improve survival. Additionally, the combination was effective to produce high numbers of Pmel-1 cells and low numbers of Tregs in tumor tissue, as further evidence of its ability to provide a notable anti-tumor effect when administered to a subject having cancer.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the invention(s) provided herein. Other aspects, advantages and modifications within the scope of the invention will be apparent to those skilled in the art to which the invention pertains.

### Materials and Methods

Recombinant human IL-2 having an amino acid sequence identical to that of aldesleukin (SEQ ID NO:3) was cloned and expressed and used to prepare the exemplary long acting IL-2Rαβ-biased agonist referred to herein as RSLAIL-2.

RSLAIL-2 refers to a composition obtainable upon following the procedures of Example 1 in PCT Int. Pat. Appl. Pub. No. WO 2015/125159, and generically refers to a composition comprising multiPEGylated forms of IL-2, wherein attachment of the PEG reagent used to form the conjugates is releasable following administration to a subject.

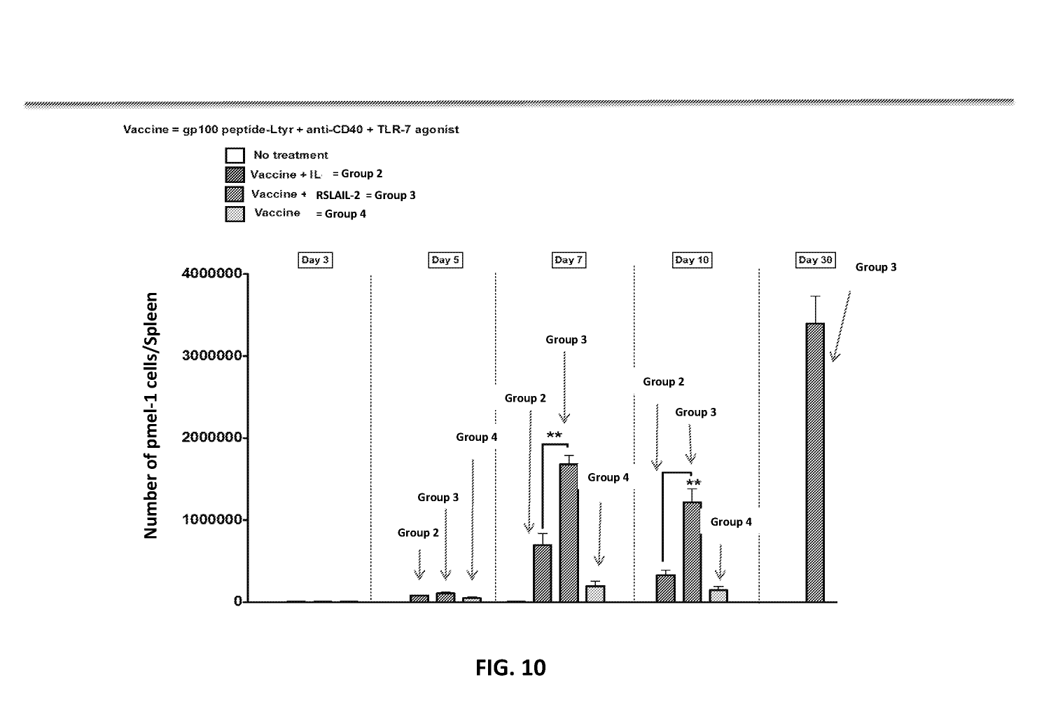

NOUS-020 Neoantigenic Vaccine: NOUS-020 constructs contain 20 non-synonymous single nucleotide variants (SNV) from the CT-26 murine tumor cell line. NOUS-020 vaccine is based on Great Apes-derived Adenovirus and MVA vaccines encoding for 20 neoantigens from a CT26 murine tumor cell line, for use in the mouse model studies described herein. The NOUS-020 insert sequence is shown in FIG. 11. For each mutation, the amino acid change is embedded in the wild type protein sequence and flanked both upstream and downstream with 12 amino acids for a total length of 25 amino acids for the neoantigen. The sequences of the protein fragments from different neoantigens are joined head to tail to form the artificial antigen fused downstream with an HA peptide sequence for the purpose of monitoring expression of the recombinant artificial protein.

### Example 1

### Pegylation of RIL-2 with MPEG2-C2-FMOC-20KD-NHS

Purified rIL-2 (106.4 mL) at 1.44 mg/ml was charged into a first vessel followed by the addition of 53.6 mL of formulation buffer (10 mM sodium acetate, pH 4.5, 5% trehalose). The pH was measured at 4.62 the temperature was measured at 21.2° C. The PEG reagent, C2-PEG2-FMOC-NHS-20K (available as described in WO 2006/138572) (13.1 g), was charged into a second vessel followed by the addition of 73.3 mL of 2 mM HCl. The resulting solution was swirled by hand for 25 minutes. Sodium borate (0.5 M, pH 9.8) was added to the first vessel to raise the pH to about 9.1 and then the contents of the second vessel containing the PEG reagent was added to the first vessel over a period of from one to two minutes. A rinse step was then performed by charging 8.1 mL of 2 mM HCl into the second vessel and adding the contents to the first vessel. For the conjugation reaction, the final rIL-2 concentration was 0.6 mg/mL, the sodium borate concentration was 120 mM, the pH was 9.1+/−0.2, the temperature was 20-22° C., and the molar ratio of PEG reagent to rIL-2, after adjustment for activity of the reagent (substitution level) was 35:1. The conjugation reaction was allowed to proceed for thirty minutes and quenched by acidification by addition of 75 mL of 2N acetic acid (to bring the pH down to approximately to 4). The product was purified by ion exchange chromatography as previously described to provide a composition of primarily 4-mers, 5-mers and 6-mers (referring to the number of PEG reagents releasably covalently attached to r-IL-2 (wherein 8-mers and higher degrees of PEGylation were removed during a washing step associated with chromatography). This composition is referred to herein as “RSLAIL-2.”

### Example 2

### Receptor-Bias of RSLAIL-2 and Related Immunotherapeutic Properties

Binding Affinity to IL-2 Receptors and Receptor Bias Related to Immunostimulatory Profile: The affinity of RSLAIL-2 to IL-2Rα and IL-2Rβ was measured directly by surface plasmon resonance (Biacore T-100) and compared to that of clinically available IL-2 (aldesleukin). Antihuman antibody (Invitrogen) was coupled to the surface of a CM-5 sensor chip using EDC/NHS chemistry. Then either human IL-2Rα-Fc or IL-2Rβ-Fc fusion protein was used as the captured ligand over this surface. Serial dilutions of RSLAIL-2 and its active IL-2 conjugates metabolites (1-PEG- and 2-PEG-IL-2) were made in acetate buffer pH 4.5, starting at 5 mM. These dilutions were allowed to bind to the ligands for 5 minutes, and the response units (RU) bound was plotted against concentration to determine EC50 values. The affinities of each isoform to each IL-2 receptor subtype were calculated as fold change relative to those of IL-2.

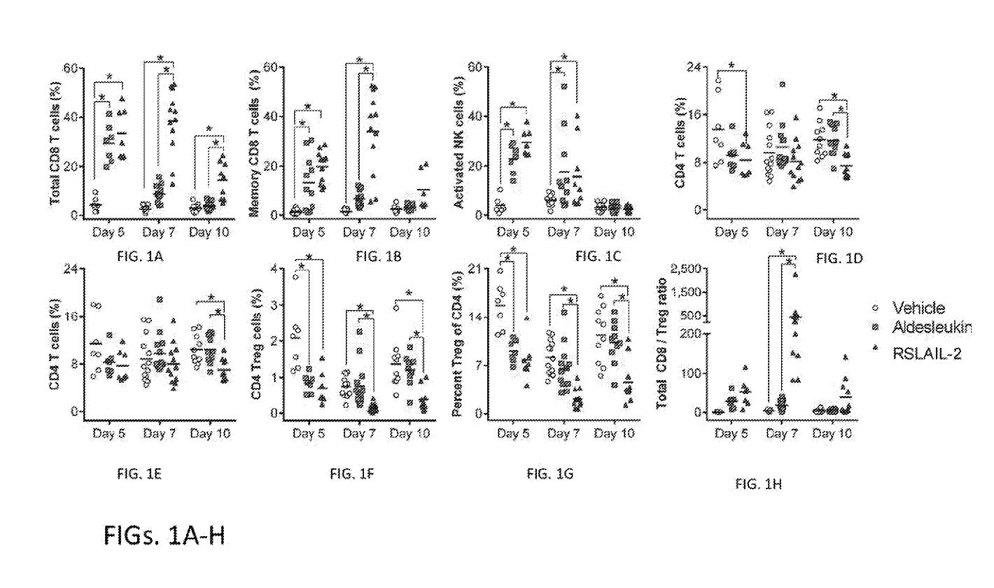

The in vitro binding and activation profiles of RSLAIL-2 suggested that PEGylation interferes with the interaction between IL2 and IL2Rα relative to aldesleukin; an investigation was carried out to determine whether these effects bias the profile of immune cell subtypes in vivo. The number of CD8 T and Treg cells in a tumor following administration of either RSLAIL-2 or IL2 is an important measure of whether pleiotropic effects of IL2 have been shifted due to conjugation of IL2 to poly(ethylene glycol) (as in RSLAIL-2) at the IL2/IL2Rα interface. To address the question, mice bearing subcutaneous B16F10 mouse melanoma tumors were treated with a single dose of RSLAIL-2 or 5 doses of aldesleukin, and immune cells in the tumor microenvironment were quantified by flow cytometry. Results are shown in FIGS. 1A-1G.

In tumors of aldesleukin-treated mice, total and memory CD8 cells were increased as a percentage of tumor-infiltrating lymphocytes; however, these effects were transient, reaching significance relative to vehicle on day 5. In contrast, significant (P<0.05) and sustained total and memory CD8 T-cell stimulation was achieved following a single RSLAIL-2 administration, with superior percentages of memory CD8 (day 7) and total CD8 (days 7 and 10) relative to aldesleukin. Both RSLAIL-2 and aldesleukin treatment resulted in increased activated natural killer (NK) cells 5 and 7 days after treatment initiation, though this effect was diminished by day 10. CD4 cell percentages of tumor-infiltrating lymphocytes were diminished following RSLAIL-2 treatment relative to vehicle on day 5. On day 10, RSLAIL-2 resulted in fewer CD4 cell percentages compared with vehicle and aldesleukin. The CD4 cell population was further analyzed for the FoxP3⁺ subset, which defines the Treg population. RSLAIL-2 administration reduced percentage of Tregs at every time point, consistent with reduced access to the IL2Rα subunit arising from the PEG chains. In contrast, Treg reduction with aldesleukin was modest achieving significance on day 5. The increase of CD8 T cells and reduction of Tregs led to a marked elevation of the CD8/Treg ratio in the tumor by day 7. The ratio of CD8/Treg for RSLAIL-2, aldesleukin, and vehicle was 449, 18, and 4, respectively, supporting preferential activation of the IL2 receptor beta over IL2 receptor alpha for RSLAIL-2.

Immunohistochemical staining was performed and confirmed that CD8 T cells were not only increased in number but were interspersed with tumor cells. These results indicate RSLAIL-2 is effective to induce a more robust in vivo memory effector CD8 T-cell response than seen with unmodified IL-2 (aldesleukin), without a commensurate stimulation of Tregs in tumor, consistent with an in vitro IL2Rβ-biased binding profile. That is to say, RSLAIL-2 is effective to preferentially activate and expand effector CD8+T- and NK-cells over Tregs.

### Example 3

### Tumor Exposure of RSLAIL-2

The objective of this study was to evaluate the antitumor activity of RSLAIL-2 in C57BL/6 mice implanted with B16F10 melanoma cells when compared to aldesleukin.

C57BL/6 mice were implanted subcutaneously into the right flank with B16F10 melanoma cells (1×10⁶ per animal). Seven days after implantation, when tumors measured 200 mm³, animals were administered RSLAIL-2 (2 mg/kg×1) or aldesleukin (3 mg/kg daily×5). Tumors were harvested (n=4 per observation time), homogenized in ice-cold PBS containing protease inhibitor (Roche) and 0.25% acetic acid, and centrifuged to obtain supernatant. To quantify RSLAIL-2 levels in tumor tissue, PEG was released from IL2 by incubating the supernatant in a pH 9 buffer at 37° C. overnight. IL2 was measured by sandwich ELISA specific for human IL2. To calculate AUC, data were fit with Pheonix WinNonLin using a noncompartmental model. AUC after aldesleukin was estimated on the basis of day 1 AUC multiplied by 5.

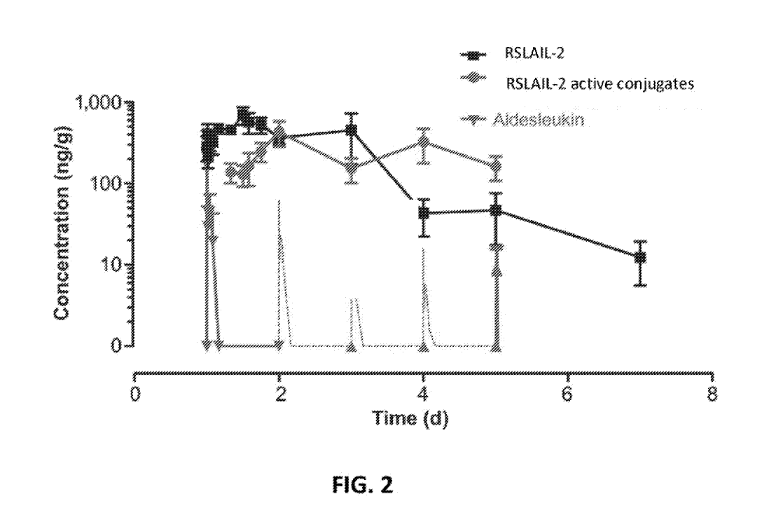

As shown in FIG. 2, tumor aldesleukin levels rapidly reached Cₘₐₓ and then rapidly declined, leading to <4 ng/g concentrations 24 hours after each dose and a daily AUC of 0.09±0.02 μg/hour/g. In contrast, RSLAIL-2 was detectable in tumors for up to 8 days after a single dose achieving an AUC of 30±6.9 μg/hour/g. On the basis of AUC, a single dose of RSLAIL-2 led to a 67-fold higher exposure compared with 5 daily doses of aldesleukin, even though 7.5 fewer IL2 equivalents were dosed using RSLAIL-2 (3 mg/kg daily×5=15 mg/kg vs. 2 mg/kg). Thus, normalizing exposure on the basis of IL2 equivalents, RSLAIL-2 achieved a 500-fold increased exposure relative to aldesleukin. The active conjugated IL-2 form of RSLAIL-2 (2-PEG-IL2 and 1-PEG-IL2 together) was also quantified and remained detectable in tumor for up to 5 days yielding an AUC of 23±4.4 μg/g. Hence, exposure to active conjugated IL2 was 50-times higher compared with aldesleukin, translating to 380 times increased exposure relative to an equivalent dose of aldesleukin. The tumor exposure of RSLAIL-2 thus allowed dosing once every 9 days in mice compared with twice daily for two 5-day cycles for aldesleukin.

### Example 4

### Evaluation of the Effectiveness of RSLAIL-2 in Improving Response to an Exemplary Vaccine, GP100, in a Murine B16 Melanoma Model

Studies were conducted to determine whether RSLAIL-2 could effectively promote expansion and function of vaccination-induced, tumor specific effector CD8+ T cells using a murine B16 melanoma model. The study compared the ability of both RSLAIL-2 and unmodified IL-2 to enhance the therapeutic efficacy of an illustrative peptide vaccine.

Initiation of the study commenced 7 days after inoculation of 300,000 B16 wild type cells/site. In the study, naïve gp100-specific TCR transgenic pmel-1 CD8+ T cells were adoptively transferred into C57BL/6 mice bearing established subcutaneous B16 tumors, followed by vaccination with (i) a vaccine formulation containing the GP-100 (glycoprotein 100) peptide vaccine (50 μg/mouse), an anti-CD40 mAb (50 μg/mouse), and a TLR-7 agonist, R848 (Resiquimod, an imidazoquinoline, 5 mice/pack) alone or (ii) in combination with RSLAIL-2 (0.2 mg/kg based on IL-2) or (iii) in combination with either high dose or low dose unmodified IL-2 (aldesleukin). Mice then received single dose of RSLAIL-2 or IL-2 (high dose) every 8 days. Treatment groups were as follows:

TABLE 1
Treatment Groups

| | |
|---|---|
| Group 1 | No treatment |
| Group 2 | IL-2 high dose: (100,000 IU × 5 doses at Day 0, Day 1, |
| | and Day 2); repeat cycle every 8 days |
| Group 3 | IL-2 low dose: 62,500 IU/mouse/every day |
| Group 4 | RSLAIL-2: 0.2 mg/kg based on protein |
| Group 5 | GP100 peptide/anti-CD40/TLR-agonist (vaccine cocktail alone) |
| Group 6 | GP100 peptide/anti-CD40/TLR-agonist + |
| | IL-2 high dose (100,000 IU × 5 doses at Day 0, Day 1, |
| | and Day 2); repeat cycle dose (100,000 IU × 5 doses) every |
| | 8 days |
| Group 7 | GP100 peptide/anti-CD40/TLR-agonist + IL-2 low dose |
| | (62,500 IU/mouse/every day) |
| Group 8 | GP100 peptide/anti-CD40/TLR-agonist + RSLAIL-2 |
| | (0.2 mg/kg) every 8 days |

Tumor growth, survival and T cell response in blood was monitored, and localization of effector pmel-1 CD8+ T cells and CD4+Foxp3+ Tregs in tumor and spleen were analyzed. T cell response was measured at Day 5, Day 7, Day 12, Day 15 and Day 20 and throughout course of treatment. FIGS. 3A-3H are plots showing tumor size (mm²) over the course of treatment for each of Groups 1-8, respectively. As shown in FIG. 3H, the combination of RSLAIL-2 and the illustrative peptide vaccine, formulated as a cocktail, was particularly effective in delaying tumor growth when compared to the other treatment groups. FIG. 4 is a graph showing average tumor size (mm²) over the course of treatment for each of the study groups for ease of comparison. As shown in FIG. 4, RSLAIL-2, an illustrative long acting IL-2Rαβ-biased agonist, when administered following vaccination, was effective to significantly delay tumor growth in the mouse model employed and achieved a markedly improved response when compared to vaccination alone or vaccination accompanied by administration of either high dose IL-2 or low dose IL-2. Turning to FIG. 4, it can be seen that, for example, after approximately 38 days of treatment, the average tumor size in the vaccination/RSLAIL-2 treatment group was approximately 25 mm², while the average tumor size in the closest treatment group (in terms of effectiveness in slowing tumor growth), vaccination/IL-2 low dose, was approximately 125 mm²—a striking difference illustrating the superior ability of an IL-2Rαβ-biased agonist such as RSLAIL-2, when accompanying vaccine therapy, to improve the therapeutic response.

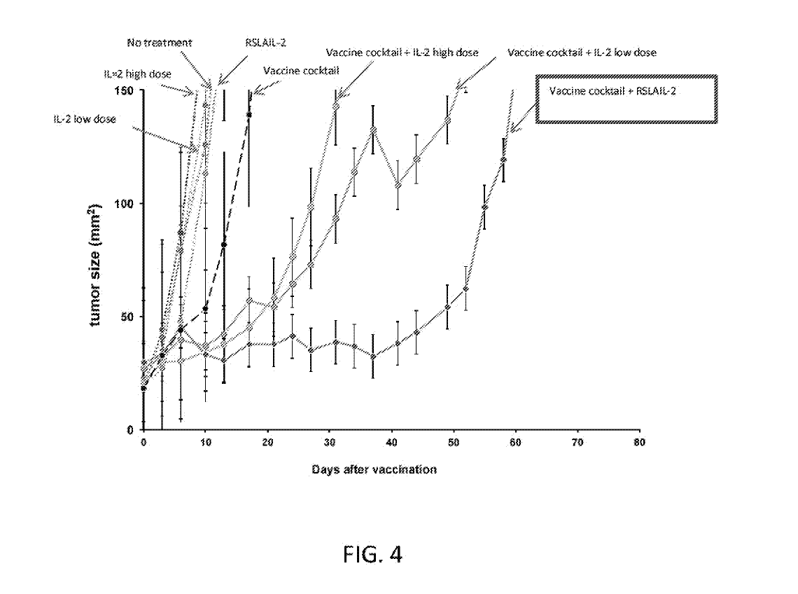

FIG. 5 is a plot associated with gp100-specific T cell function, i.e., demonstrating IFN-g+ Tcells (expressed as a percentage of pmel-1) over the course of treatment for the various treatment groups described above. The plot indicates a stable and persistent IGN-g+ T cell (pmel-1) response at above 90% extending to about 40 days post vaccination for the GP100/anti-CD40/TRL-7 agonist/RSLAIL-2 treatment group; the vaccine/RSLAIL-2 combination therapy reached and maintained the highest percentage of IFN-g+ Tcell (pmel-1) response over the other treatment groups. Additionally, the vaccine/RSAIL-2 combination therapy-induced IGN-g+ T cell (pmel-1) response was slower to decline than in the other treatment groups.

FIG. 6 is a plot demonstrating percent survival for each of the treatment groups. Consistent with the plots showing tumor size over the course of treatment (FIGS. 3A-3H and FIG. 4), survival for the vaccine/RSLAIL-2 treatment group (GRP8) was significantly enhanced in comparison to the other treatment groups. 100% of subjects in the peptide vaccine/long acting IL-2Rαβ-biased agonist treatment group survived to about 57 days, with 50% survival at approximately 62 days; for the next closest treatment group in terms of positive response to therapy, i.e., the peptide vaccine/low dose IL-2 group, 100% survival was observed to approximately 32 days, with 50% survival at about 48 days.

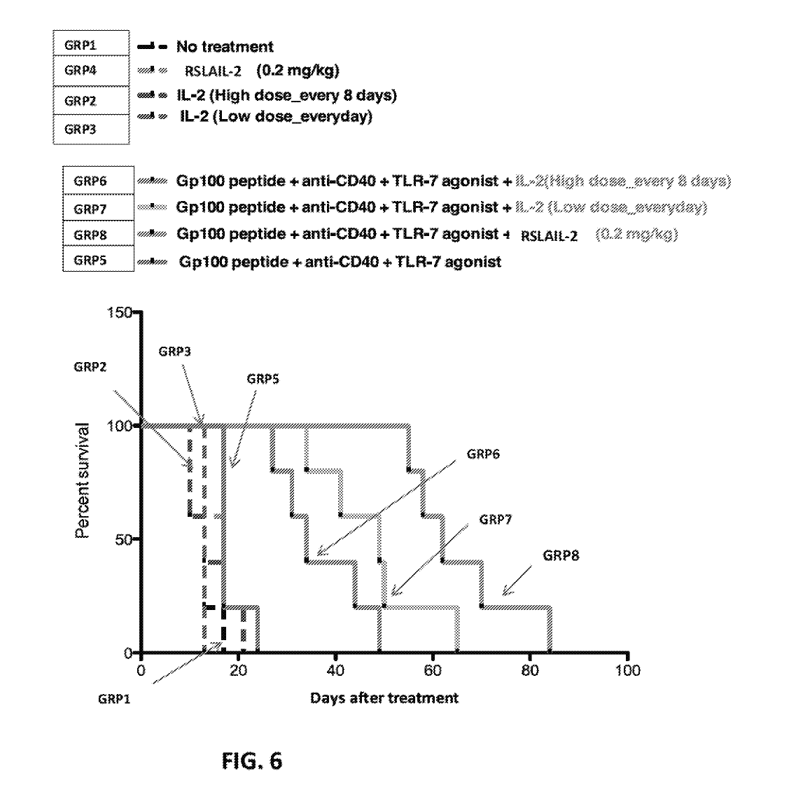

FIG. 7 is a plot demonstrating percent pmel-cells (expressed as a percentage of total CD8+ T cells) for each of the treatment groups over the course of treatment. RSLAIL-2, when combined with the GP-100 vaccine, exhibited a notably elevated pmel-1 response when compared to both high dose and low dose IL-1 treatment coupled with peptide vaccine therapy.

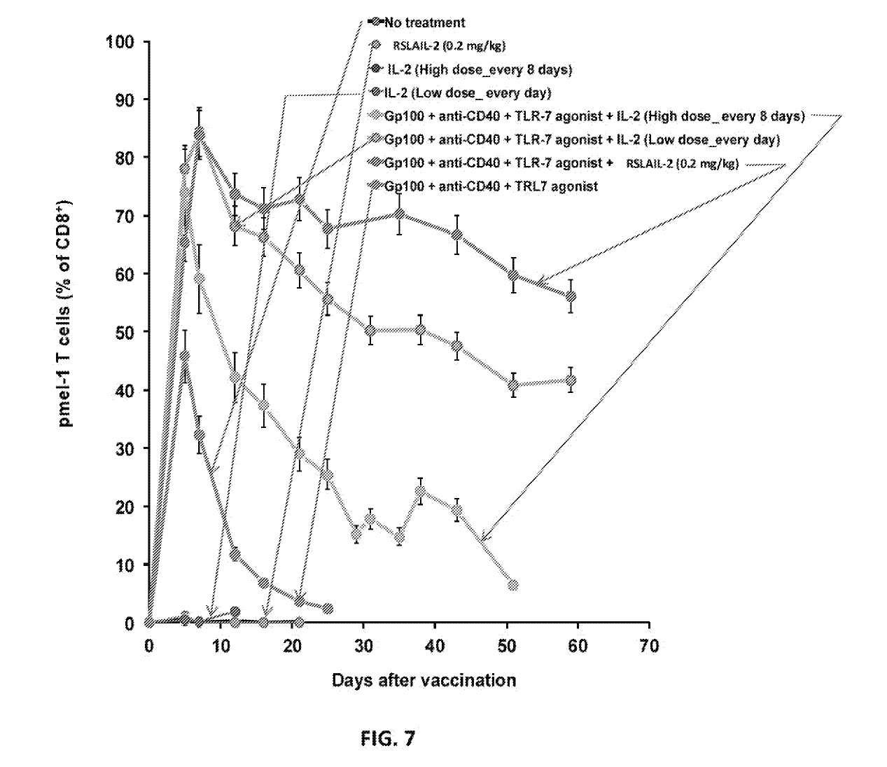

FIG. 8 is a plot showing regulatory T cells, i.e., CD25+Foxp3+ T cells, expressed as a percentage of CD4 cells over the course of treatment in C57BL/6 mice bearing established subcutaneous B16 tumors for each of the study groups described in Example 4. As can be seen from the plot, the percentage of RSLAIL-2-induced regulatory T cells decreases rapidly around the end of each dosing cycle.

RSLAIL-2, an illustrative long acting IL-2Rαβ-biased agonist, demonstrated notable synergy with vaccination, potently suppressing tumor growth and significantly improving survival of mice when compared to vaccination alone or accompanied by administration of unmodified (i.e., non-long acting) IL-2, administered in both high dose and low dose treatment modalities. RSLAIL-2 additionally enhanced pmel-1 CD8+ T cell numbers and decreased numbers of immune-suppressive Tregs in tumor. RSLAIL-2 was effective to stably maintain a high ratio of pmel-1 CD8+ T cells over Tregs in tumors for over 30 days. Despite the induction of very strong CD8+ T cell responses and anti-tumor activity, no gross toxicity was observed.

### Example 5

### Further Evaluation of the Effectiveness of RSLAIL-2 in Improving Response to an Exemplary Vaccine, GP100, in a Murine B16 Melanoma Model—Analysis of Pmel and Tregs in Tumor, Spleen and Blood

Studies were conducted to investigate the impact of RSLAIL-2 when administered in combination with an exemplary peptide vaccine on the localization of effector CD8+ T cells and Tregs to tumor and spleen using a murine B16 melanoma model as described in Example 4 above.

In the study, naïve gp100-specific TCR transgenic pmel-1 CD8+ T cells were adoptively transferred into C57BL/6 mice bearing established subcutaneous B16 tumors, followed by vaccination with (i) a cocktail containing GP-100, a glycoprotein-100 peptide vaccine (50 μg/mouse), an anti-CD40 mAb (50 μg/mouse), and a TLR-7 agonist, R848 (Resiquimod, an imidazoquinoline, 5 mice/pack) alone or (ii) in combination with RSLAIL-2 (0.2 mg/kg based on IL-2) or (iii) in combination with high dose IL-2 (aldesleukin). Mice then received a single dose of RSLAIL-2 or IL-2 (high dose) every 8 days. Treatment groups were as follows:

TABLE 2
Treatment Groups

| | |
|---|---|
| Group 1 | No treatment |
| Group 2 | GP100 peptide/anti-CD40/TLR-agonist + IL-2 high dose |
| | (100,000 IU × 5 doses at Day 0, Day 1, and Day 2); |
| | repeat cycle every 8 days |
| Group 3 | GP100 peptide/anti-CD40/TLR-agonist + RSLAIL-2 |
| | (0.2 mg/kg) every 8 days |
| Group 4 | GP100 peptide/anti-CD40/TLR-agonist (vaccine cocktail alone) |

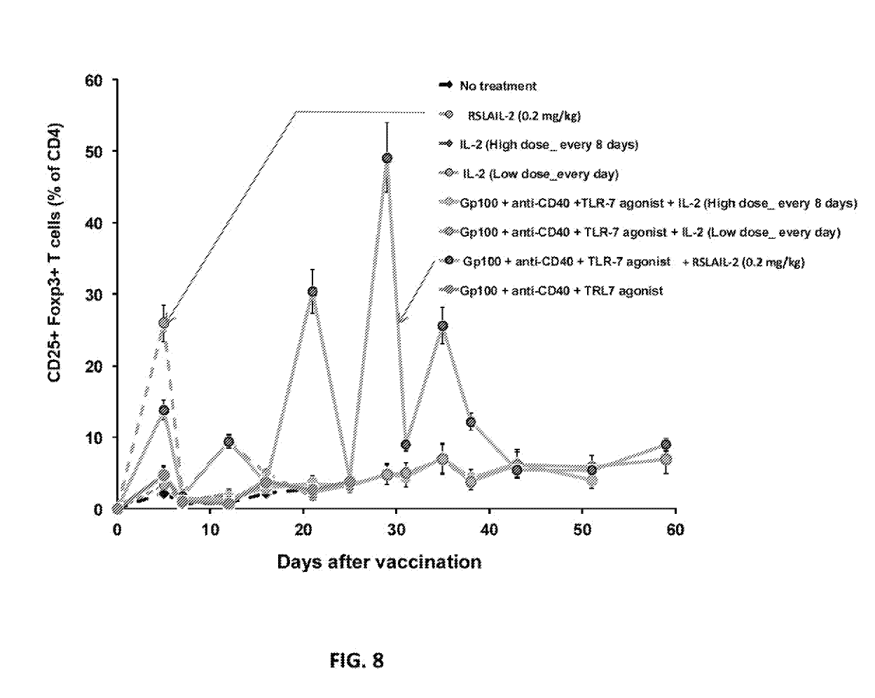

The tumor and spleen cell samples were collected and first treated with a fixable viability indicator and then stained for viable immune cells. The total amount of live immune cells were counted for each of the samples and used for gating/collecting total events for the subject cell types. The primary cell counts were derived from the summarized raw data of the flow cytometer reading and analyzed. FIG. 9 is a bar graph indicating numbers of Thy1.1+ pmel-1 cells/gram of tumor at each of days 5, 7, 10 and 30 for treatment groups 2, 3 and 4. As can be seen, when comparing vaccine treatment accompanied by administration of either high dose IL-2 or RSLAIL-2, RSLAIL-2 is effective to induce a significantly higher and stable Pmel-1 response in tumor tissue than is IL-2. FIG. 10 is a bar graph indicating numbers of Thy1.1+ pmel-1 cells/gram of spleen at each of days 5, 7, 10 and 30 for treatment groups 2, 3 and 4. As can be seen, when comparing vaccine treatment accompanied by administration of either high dose IL-2 or RSLAIL-2, in a fashion similar to the tumor microenvironment, RSLAIL-2 is effective to induce a significantly higher and stable Pmel-1 response in spleen than is IL-2. Similar to the results described in Example 4 above, RSLAIL-2 effectively mediated reduction of regulatory Tcells (Tregs) at day 7 and maintained minimal numbers of Tregs in the tumor extending until at least day 30. Based upon evaluation of various immune cell types over the course of treatment (Tregs and non-Tregs), the peptide vaccine when combined with RSLAIL-2, but not with IL-2, produced higher Pmel to Tregs ratio in the tumor as well as in the spleen. In sum, based upon these data, it appears that the exemplary IL-2Rαβ-biased agonist, RSLAIL-2, is markedly better than IL-2 in stably maintaining high numbers of Pmel-1 cells and low Tregs in tumor tissue and over a longer period of time. Further, RSLAIL-2 specifically inhibited accumulation of Tregs to the tumor, and promoted maintenance of a high ratio of Pmel to Tregs in tumor tissue up to day 30 of treatment.

### Example 6

### Immunogenicity OF NOUS-020 GAd Vaccine

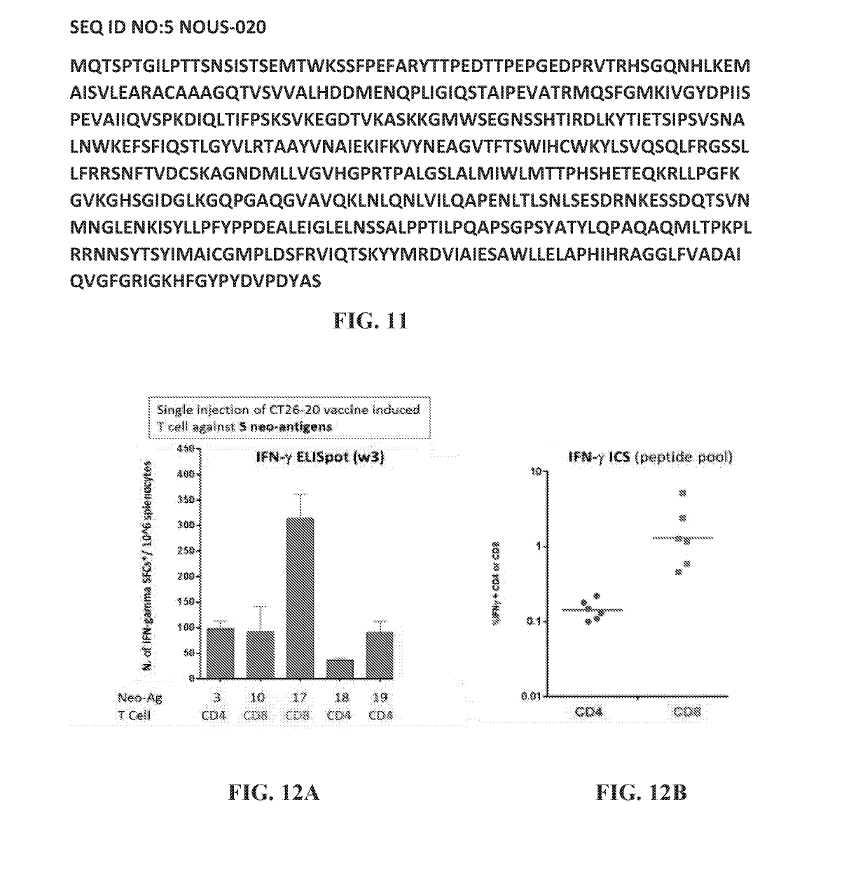

The immunogenicity of the NOUS-020 GAd vaccine was evaluated in BALB/c inbred mice after single intramuscular immunization at dose of 5×10⁸ viral particles (vp). Splenocytes were collected three weeks post-immunization and tested by IFN-γ ELISpot by stimulating cells in the presence of synthetic peptides corresponding to each neoantigen vaccine encoded. Negative-control cultures included cells stimulated with culture medium alone in presence of peptide diluent dimethyl sulfoxide (DMSO). Immune responses (number of T cells producing IFN-γ per million splenocytes) are shown in the related figures. Responses were considered positive if the mean of antigen wells was greater than 15 SFC/10⁶ PBMC and exceeded by 3-fold the background value of DMSO wells. Quality of T cell responses (CD4 and CD8) was measured by Intracellular IFN-γ cytokine staining with a pool of 5 neo-antigens that were immunogenic in the ELISpot assay. Immune responses (number of T cells producing IFN-γ per million splenocytes) are shown in FIGS. 12A and 12B. As can be seen, NOUS-020 GAd vaccine induces CD4 and CD8 T cells.

### Example 7

### Immunogenicity of NOUS-020 GAd-MVA Vaccine

The immunogenicity of the NOUS-020 GAd-MVA vaccine was evaluated in prime-boost studies. BALB/c inbred mice were primed with GAd (dose of 5×10⁸ viral particles) and then boosted with MVA (10⁷ pfu) at week 4. Vaccine-induced responses were measured one week post boost by IFN-γ ELISpot stimulating spleen cells with a pool of 20 vaccine encoded neoantigens. Negative-control cultures included cells stimulated with culture medium alone in the presence of the peptide diluent, dimethyl sulfoxide (DMSO).

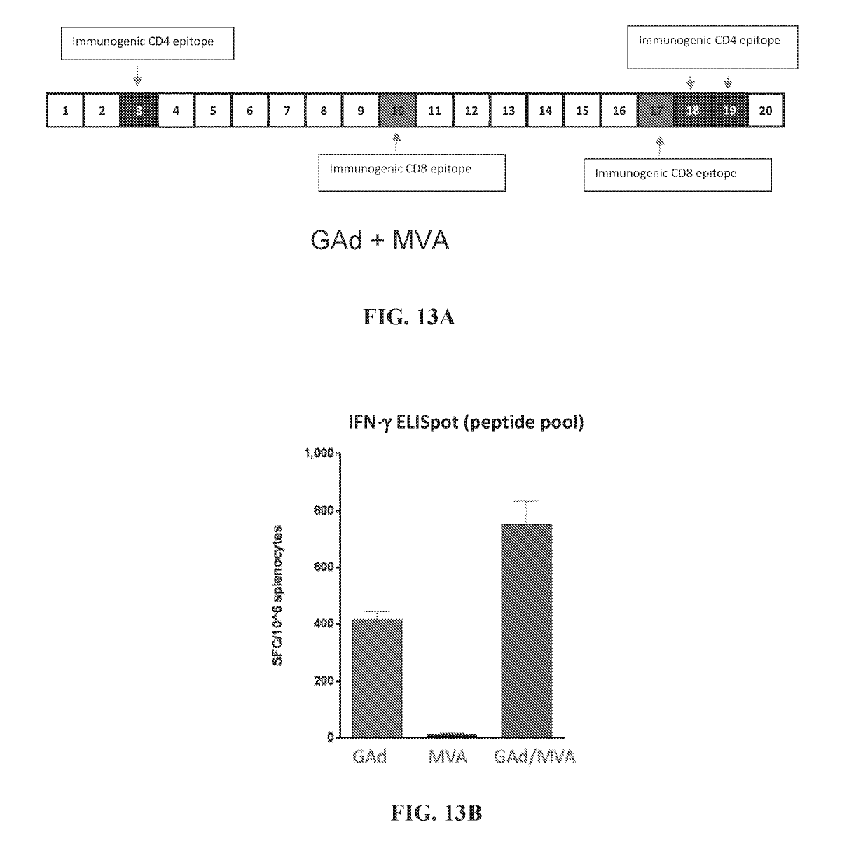

Immune responses (number of T cells producing IFN-γ per million splenocytes) are shown in FIGS. 13B and 13C. Responses were considered positive if the mean of antigen wells was greater than 15 SFC/10⁶ PBMC and exceeded by 3 fold the background value of DMSO wells. Quality of T cell responses (CD4 and CD8) was measured by Intracellular IFN-γ cytokine staining with a pool of 5 neo-antigens resulted immunogenic for ELISpot assay. The quality of T cell responses (CD4 and CD8) was measured by Intracellular IFN-γ cytokine staining with a pool of top 5 neo-antigens resulted immunogenic for ELISpot assay. As shown, the neoantigenic vaccine induced a response for CD4 and CD8 T-cells. FIG. 13A provides a schematic of constructs showing the neoantigens that induce a CD8 and CD4 response. FIG. 13B provides an analysis of T cell responses measured post GAd/MVA immunization in naive mice by IFN-γ ELISpot on pool of 20 vaccine encoded neo-antigens.

### Example 8

### Combination Treatment with NOUS-020 GAd-CT26 Neoantigenic Vaccine and RSLAIL-2 in a Murine CT26 Tumor Model (Early Therapeutic Setting)

The therapeutic efficacy of concomitant administration (NOUS-020 GAd vaccine and RSLAIL-2 at Day 0) and subsequent administration (GAd Day 0, and RSLAIL-2, Day 7) of NOUS-020 vaccine and RSLAIL-2 was evaluated on CT26 tumor growth in BALB/c mice.

BALB/c mice were injected with CT26 colon carcinoma cells at Day-3. Three days later (Day 0), mice were treated with (i) NOUS-020 GAd vaccine alone (intramuscularly, at a dose of 5×10⁸ viral particles), (ii) RSLAIL-2 alone (intravenously, 0.8 mg/kg, q9x3), or a combination of NOUS-020 GAd vaccine and RSLAIL-2 administered either (iii) concomitantly at Day 0 or (iv) with a sequential administration regimen with NOUS-020 GAd vaccine administered at Day 0 and RSLAIL-2 administered at Day 7.

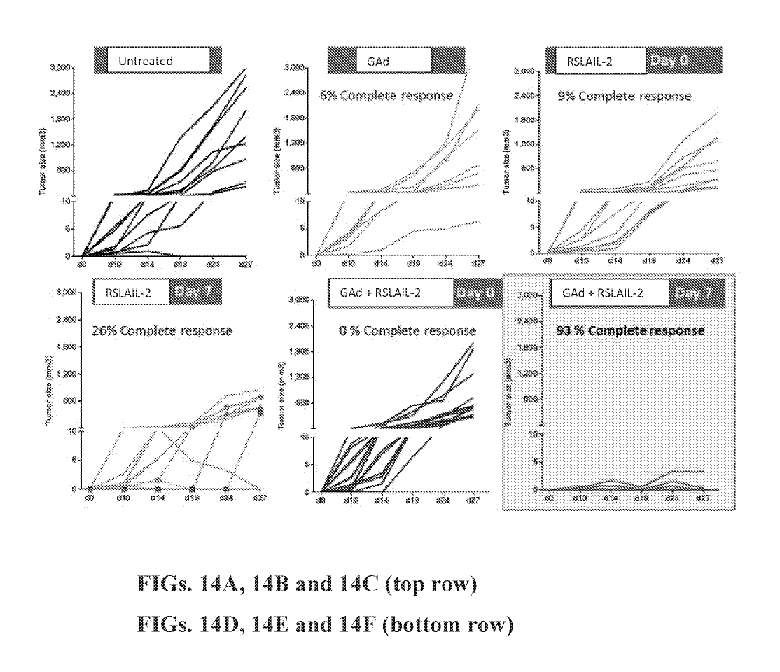

Tumor volumes were recorded over time for each treatment group. Results are shown in FIGS. 14A-14F. FIG. 14A provides results for the control group (untreated); FIG. 14B demonstrates volume of CT26 tumors in mice treated with GAd vaccine alone; FIGS. 14C and 14D demonstrate volume of CT26 tumors in mice treated with RSLAIL-2 (administered at either day 0 or 7, respectively) and concomitant at Day 0 (FIG. 14E) or sequential administration (FIG. 14F) of RSLAIL-2 and GAd, respectively.

As can be seen from the figures, administration of RSLAIL-2 on day 7 notably improved the efficacy of the NOUS-020 GAd vaccine in an early therapeutic setting (i.e., prior to growth of a sizable tumorous mass).

To further explore various treatment regimens for administering NOUS-020 GAd vaccine and RSLAIL-2 in combination, different treatment intervals were explored.

BALB/c mice were challenged with CT26 tumor cells and 3 days post challenge (day 0) they received NOUS-020 GAd vaccine (day 0, 5×10⁸ viral particles) or a combination of NOUS-020 GAd administered at day 0 and RSLAIL-2 administered either at day 3, 5, or 7. Tumor growth was monitored over time in the various treatment groups. Table 3 below shows the percentage of tumor-free mice at the end of the study for each treatment modality.

TABLE 3
Dosing Regimen Versus Complete Response

| | Percent Tumor Free Mice |
|---|---|
| Regimen | (Percent Complete Response) |
| NOUS-020 GAd | 6% |
| NOUS-020 GAd + RSLAIL-2 (Day 3) | 28% |
| NOUS-020 GAd + RSLAIL-2 (Day 5) | 70% |
| NOUS-020 GAd + RSLAIL-2 (Day 7) | 80% |

Based upon the data in Table 3, it appears that the interval between GAd vaccination and single dose administration of RSAIL-2 affects synergic activity. Based on the data above, it appears that RSAIL-2 is preferably first administered at more than than 5 days following vaccination, for example, at 6 days, or at 7 days, or at 8 days, or at 9 days or at 10 days or more following vaccination.

### Example 9

### Combination Treatment of Established Tumors with NOUS-020 GAd-MVA CT26 Neoantigenic Vaccine and RSLAIL-2 in a Murine CT26 Tumor Model

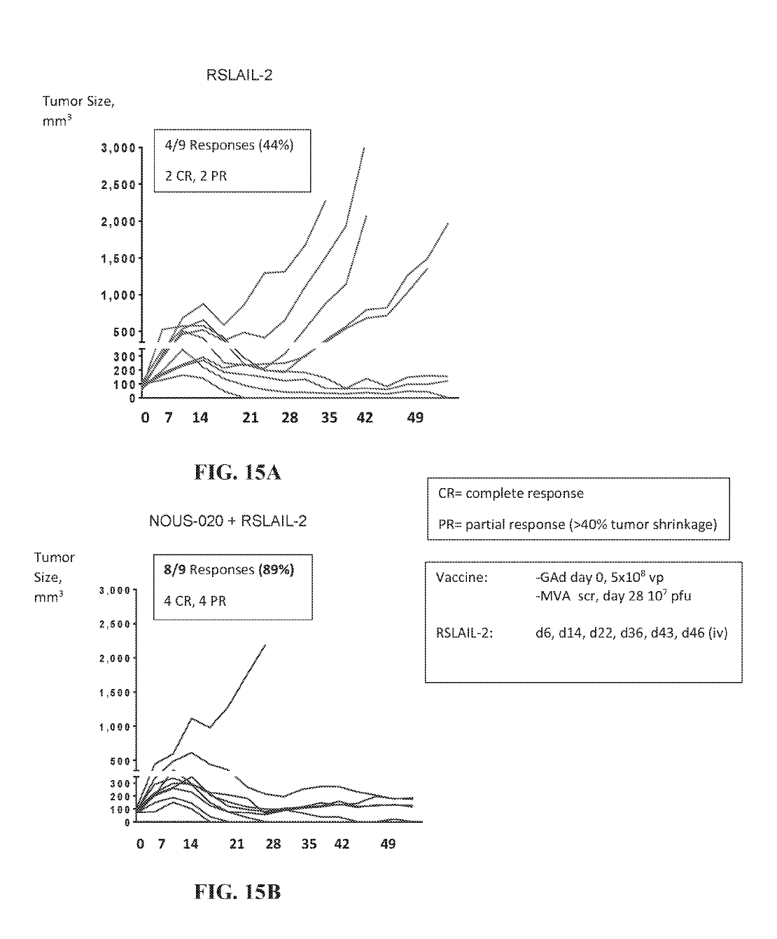

BALB/c mice were challenged with CT26 cells. After a week, mice having a tumor mass of 100 mm³ were randomized into 2 groups (day 0), one group receiving RSLAIL-2 alone, the second group receiving a combination of NOUS-020 and RSLAIL-2, respectively, administered at day 0 (5×10⁸ viral particles) and day 6 (intravenous, 0.8 mg/kg). Administration of RSAIL-2 was repeated at day 14, day 22, day 36, day 43, and day 46. Boost with MVA for the group receiving the combination treatment was performed at day 28, with intramuscular injection of MVA at a dose of 10⁷ pfu. Tumor volumes were monitored over time. Results are shown in FIGS. 15A (RSAIL-2 only) and 15B (NOUS-020 and RSAIL-2). The group administered RSLAIL-2 alone had a 44% response rate with 2 complete responders and 2 partial responders (partial response=greater than 40% tumor shrinkage, but not complete disappearance of tumor). In contrast, the group administered a combination of NOUS-020 and RSLAIL-2 as described above had an 89% response to treatment, with 4 complete responders and 4 partial responders.

These results demonstrate that combined therapy with the illustrative mouse neoantigenic NOUS-020 cancer vaccine and RSLAIL-2 is effective in treating mice with established tumors.

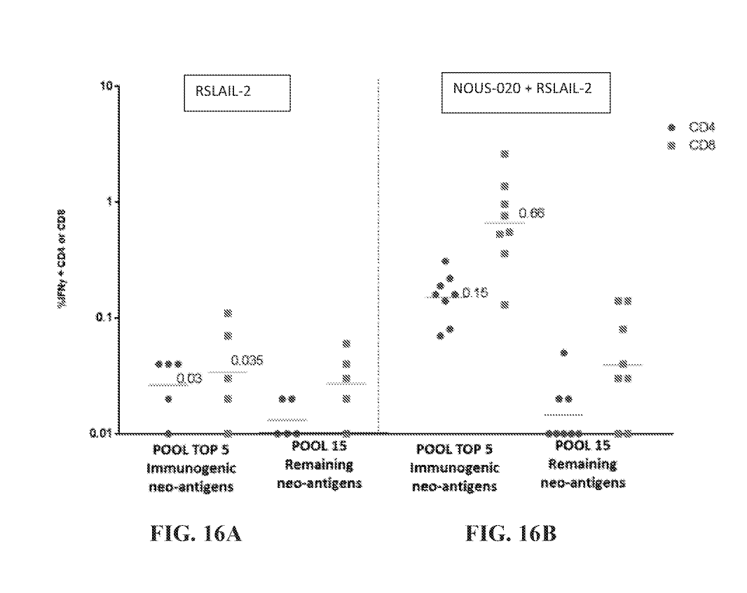

Responder mice from the combination group NOUS-020 and RSLAIL-2 or RSLAIL-2 only were sacrificed at day 54. An assessment of antigen-specific T cell responses was performed by intracellular IFN-γ staining on spleen cells stimulated in the presence of two separate peptide pools: a pool of top 5 immunogenic neo-peptides and a second pool containing the remaining 15 vaccine encoded neo-peptides. Results are shown in FIGS. 16A (RSLAIL-2 only) and 16B (NOUS-020 and RSLAIL-2).

### Example 10

### Anti-Tumor Effect of RSLAIL-2 Combined with Vaccination in a Murine C26 Colon Carcinoma Model

A study was conducted to investigate the anti-tumor immune response to vaccination using an illustrative single antigen peptide vaccine (AH1 peptide) combined with administration of RSLAIL-2 in a C26 colon carcinoma model in BALB/c mice (5 mice per group).

Initiation of the study (Day 0) commenced 4 days after inoculation of 1×10⁶ CT26 wild type cells per mouse. Study groups were as follows:

Group 1 (untreated);

Group 2: RSLAIL-2 only, 0.8 mg/kg, administered every 8 days;

Group 3: AH1 vaccine formulation only. Vaccine included AH1 peptide (immunodominant CD8 epitope derived from gp70₍₄₂₃₋₄₃₁₎ expressed in CT26, amino acid sequence SPSYVYHQF (Huang, A., et al., Immunology. Proc. Natl. Acad. Sci, USA, 93, 9730-9735 (1996), 25 μg/mouse), anti-αCD-40 mAB (50 μg/mouse), and a toll-like receptor 7 agonist, imiquimod, 5 mice/pack), administered at Day 5 and Day 13;

Group 4: AH1 vaccine (AH1 peptide, gp70₍₄₂₃₋₄₃₁₎, 25 μg/mouse/anti-αCD-40 mAB (50 μg/mouse)/imiquimod, 5 mice/pack) and RSLAIL-2 (0.8 mg/kg), administered at Day 4, and Day 12.

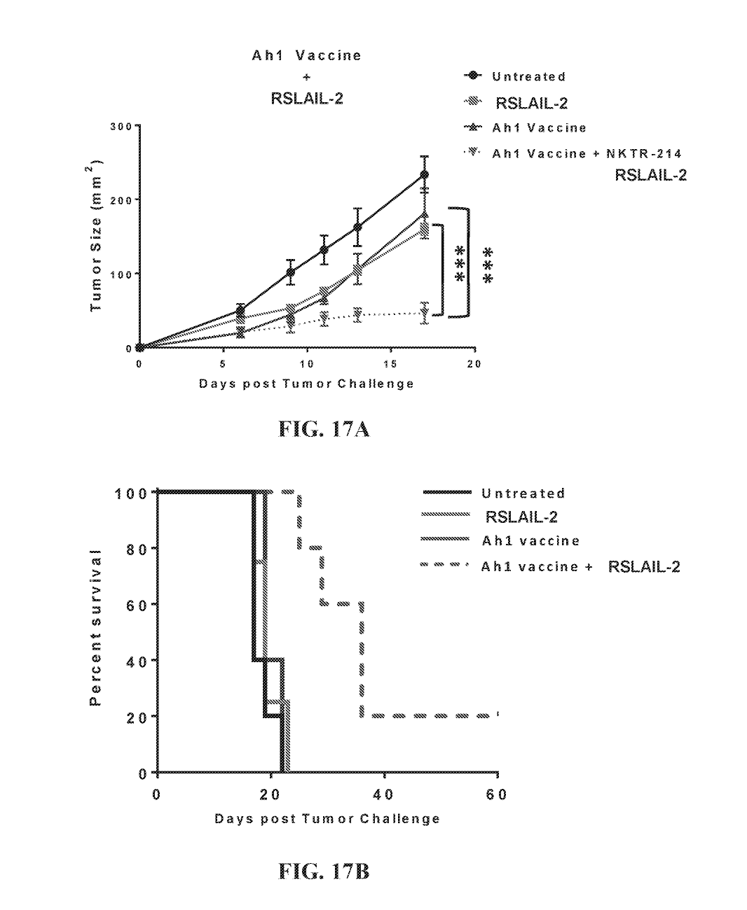

Tumor volumes were monitored over time. Results are shown in FIGS. 17A and 17 B. As shown in the figures, both the AH-1 vaccine and RSLAIL-2 when administered alone delayed tumor growth, however when administered in combination, a significant anti-tumor effect was observed (20% survival rate).

Results: In a mouse model of colon cancer, the illustrative AH-1 single antigen peptide vaccine, when administered in combination with RSLAIL-2, notably delayed tumor growth and improved survival when compared to each of the AH-1 vaccine and RSLAIL-2, when administered alone.

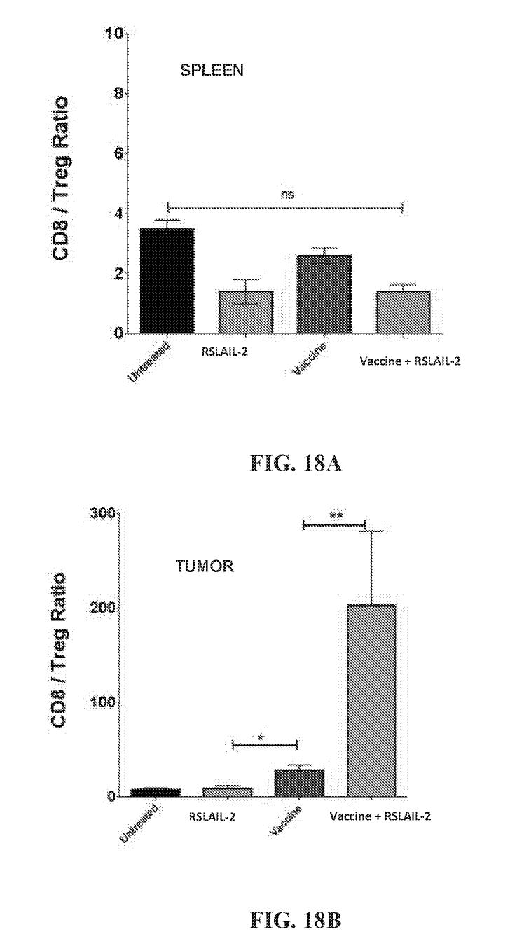

A separate study was carried out to determine localization of tumor CD8+ T cells versus Tregs in tumor and spleen of CT26-tumor treated mice treated with either the AH1 peptide vaccine, RSLAIL-2, or a combination of both, as described above. Tumor and spleen cell samples were collected at Day 7, and first treated with a fixable viability indicator and then stained for viable immune cells. The total amount of live immune cells were counted for each of the samples and used for gating/collecting total events for the subject cell types. The primary cell counts were derived from the summarized raw data of the flow cytometer reading and analyzed. Results are shown in FIGS. 18A and 18B.

Results: As provided in the figures, vaccination coupled with administration of RSLAIL-2 led to a significantly higher ratio of CD8 T cells versus Tregs in the tumor when compared to the spleen. Thus, administration of RSLAIL-2 when combined with vaccination, is effective to induce a significantly higher and stable Pmel-1 response in tumor tissue than either vaccination alone or administration of RSLAIL-2 alone.

```
(IL-2 WITH PRECURSOR)
SEQ ID NO: 1
  MYRMQLLSCI ALSLALVTNS APTSSSTKKT QLQLEHLLLD LQMILNGINN
-20 -10 1 11 21
  YKNPKLTRML TFKFYMPKKA TELKHLQCLE EELKPLEEVL NLAQSKNFHL
  31 41 51 61 71
  RPRDLISNIN VIVLELKGSE TTFMCEYADE TATIVEFLNR WITFCQSIIS
  81 91 101 111 121
TLT
(IL-2)
SEQ ID NO: 2
                                            APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS
TLT
(aldesleukin = des-alanyl-1, serine-125 human interleukin-2)
SEQ ID NO: 3
                                              PTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFSQSIIS
TLT
(BAY 50-4798 or ″N88R mutant″, see WO99/60128)
SEQ ID NO: 4
                                            APTSSSTKKT QLQLEHLLLD LQMILNGINN
YKNPKLTRML TFKFYMPKKA TELKHLQCLE EELKPLEEVL NLAQSKNFHL
RPRDLISRIN VIVLELKGSE TTFMCEYADE TATIVEFLNR WITFCQSIIS
TLT
NOUS-020
SEQ ID NO: 5
MQTSPTGILPTTSNSISTSEMTWKSSFPEFARYTTPEDTTPEPGEDPRVTRHSGQNHLKE
MAISVLEARACAAAGQTVSVVALHDDMENQPLIGIQSTAIPEVATRMQSFGMKIVGYD
PIISPEVAIIQVSPKDIQLTIFPSKSVKEGDTVKASKKGMWSEGNSSHTIRDLKYTIETSIPSV
SNALNWKEFSFIQSTLGYVLRTAAYVNAIEKIFKVYNEAGVTFTSWIHCWKYLSVQSQLFR
GSSLLFRRSNFTVDCSKAGNDMLLVGVHGPRTPALGSLALMIWLMTTPHSHETEQKRLL
PGFKGVKGHSGIDGLKGQPGAQGVAVQKLNLQNLVILQAPENLTLSNLSESDRNKESSD
QTSVNMNGLENKISYLLPFYPPDEALEIGLELNSSALPPTILPQAPSGPSYATYLQPAQAQ
MLTPKPLRRNNSYTSYIMAICGMPLDSFRVIQTSKYYMRDVIAIESAWLLELAPHIHRAGG
LFVADAIQVGFGRIGKHFGYPYDVPDYAS
```

1. A method of administration, the method comprising administering to a subject having cancer an IL-2Rβ-activating amount of a long acting IL-2Rβ-biased agonist and a cancer vaccine, wherein the long-acting IL-2Rβ-biased agonist is administered at a dose that is less than about 0.7 mg/kg.

2. A method of enhancing the therapeutic effectiveness of a cancer vaccine, comprising administering to a subject having cancer a cancer vaccine and an IL-2Rβ-activating amount of a long-acting IL-2Rβ-biased agonist, wherein the long-acting IL-2Rβ-biased agonist is administered at a dose that is less than 0.7 mg/kg, and the administering of the long-acting IL-2Rβ-biased agonist is effective to improve the subject's response to the vaccine.

3. A method of treating cancer in a subject, comprising administering to the subject an IL-2Rβ-activating amount of a long-acting IL-2Rβ-biased agonist and a cancer vaccine in an amount effective to treat cancer, wherein the long-acting IL-2Rβ-biased agonist is administered at a dose that is less than about 0.7 mg/kg, and when evaluated in a mouse model of the cancer using equivalent amounts of the long acting IL-2Rβ-biased agonist and the vaccine, is effective to prolong survival over administration of the cancer vaccine and a non-long acting version of the IL-2 agonist by at least 15 days based upon the time delay between 50% maximum tumor growth for each of the foregoing treatments.

4. A method of inhibiting accumulation of regulatory T cells (Tregs) in a subject undergoing treatment for cancer, comprising administering to the subject an IL-2Rβ-activating amount of a long acting IL-2Rβ-biased agonist and a cancer vaccine in an amount effective to treat cancer, where when evaluated in a mouse model of cancer using equivalent amounts of the long acting IL-2Rβ-biased agonist and the vaccine, is effective to inhibit accumulation of regulatory T cells selected from the group consisting of CD4+ Tregs, CD25+ Tregs, and FoxP3+ Tregs in the tumor by an amount that is enhanced over that observed upon administration of a non-long acting IL-2Rβ-biased agonist and the vaccine.

5. The method of claim 1, wherein the vaccine is administered to the subject separately from the long acting IL-2Rβ-biased agonist.

6. The method of claim 5, wherein the vaccine is administered to the subject prior to administering the long acting IL-2Rβ-biased agonist.

7. The method of claim 1, wherein the vaccine and the long acting IL-2Rβ-biased agonist are both administered on day 1 of treatment.

8. The method of claim 1, wherein the vaccine is administered on day 1 of treatment and the long acting IL-2Rβ-biased agonist is administered at any one of days 1 to 4 of treatment.

9. The method of claim 1, wherein the subject is a human.

10. The method of claim 1, wherein the cancer is a solid cancer.

11. The method of claim 10, wherein the cancer is selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

12. The method of claim 11, wherein the cancer is a malignant melanoma.

13. The method of claim 1, wherein the long acting IL-2Rβ-biased agonist is administered at a dose in a range of less than 0.7 mg/kg to about 0.2 mg/kg.

14. The method of claim 10, wherein the administering is effective to result in a reduction in solid tumor size of at least 25% when evaluated after 1 cycle of treatment.

15. The method of claim 1, wherein the long acting IL-2Rβ-biased agonist comprises aldesleukin releasably covalently attached to polyethylene glycol.

16. The method of claim 15, wherein the long acting IL-2Rβ-biased agonist comprises aldesleukin releasably covalently attached to an average of 6 polyethylene glycol polymers.

17. The method of claim 1, wherein the vaccine is selected from an antigen vaccine, a whole cell vaccine, a dendritic cell vaccine, and a DNA vaccine.

18. The method of claim 17, wherein the vaccine is an allogenic vaccine.

19. The method of claim 17, wherein the vaccine is an autologous vaccine.

20. The method of claim 17, wherein the vaccine is an antigen vaccine.

21. The method of claim 20, wherein the antigen vaccine comprises a tumor-specific antigen.

22. The method of claim 21, wherein the tumor-specific antigen is selected from a cancer-testis antigen, a differentiation antigen, and a widely-occurring over-expressed tumor associated antigen.

23. The method of claim 20, wherein the vaccine comprises a neoantigen.

24. The method of claim 1, wherein the vaccine is administered in the form of a composition comprising one or more adjuvants.

25. A kit comprising an IL-2Rβ-activating amount of a long acting IL-2Rβ-biased agonist and a vaccine, accompanied by instructions for use in treating a subject having cancer.

26. (canceled)

27. (canceled)

28. (canceled)

29. (canceled)

30. The kit of claim 25, wherein both the long-acting IL-2Rβ-biased agonist and the vaccine are in solid form.

31. (canceled)

32. (canceled)

33. The kit of claim 25, wherein both the long acting IL-2Rβ-biased agonist and the vaccine are in a solid form suitable for reconstitution in an aqueous diluent.
